(19) United States
(12) Patent Application Publication
(10) Pub. No.: US 2006/0247165 A1
(43) Pub. Date: Nov. 2, 2006

(54) Methods and compositions for encapsulation of cells

(76) Inventors: Samuel I. Stupp, Chicago, IL (US); John Allen Kessler, Chicago, IL (US)

Correspondence Address: MEDLEN & CARROLL, LLP, Suite 350, 101 Howard Street, San Francisco, CA, 94105, US

(21) Appl. No.: 11/337,316

(22) Filed: Jan. 23, 2006

Related U.S. Application Data
(60) Provisional application No. 60/645,668, filed on Jan. 21, 2005.

(51) Int. Cl. A61K 38/18; A61K 38/08

(52) U.S. Cl. 514/12; 514/17

(57) ABSTRACT

The present invention relates to methods and compositions for altering (e.g., augmenting or stimulating) differentiation and growth of cells (e.g., neural progenitor cells and neurons). In particular, the present invention relates to compositions comprising one or more self-assembling peptide amphiphiles (e.g., in solution or that generate (e.g., self-assemble into) nanofibers (e.g., that are able to encapsulate cells and promote cellular differentiation (e.g., neurite development))) and methods of using the same. Compositions and methods of the present invention find use in research, clinical (e.g., therapeutic) and diagnostic settings.

This application claims priority to U.S. Provisional Patent Application Ser. No. 60/645,668, filed Jan. 21, 2005, hereby incorporated by reference in its entirety.

This work was supported in part by the U.S. Department of Energy (grant DE-FG02-00ER45810/A001), NIH (grants NS20778, NS20013, and NS34758), and NSF (DMR-010-8342). The government may have certain rights in the invention.

### FIELD OF THE INVENTION

The present invention relates to methods and compositions for altering (e.g., augmenting or stimulating) differentiation and growth of cells (e.g., neural progenitor cells and neurons). In particular, the present invention relates to compositions comprising one or more self-assembling peptide amphiphiles (e.g., in solution or that generate (e.g., self-assemble into) nanofibers (e.g., that are able to encapsulate cells and promote cellular differentiation (e.g., neurite development))) and methods of using the same. Compositions and methods of the present invention find use in research, clinical (e.g., therapeutic) and diagnostic settings.

### BACKGROUND OF THE INVENTION

Although 3-dimensional scaffolds exist for storing or attracting cells, they are often deficient in several areas. For example, cells induced to differentiate often differentiate into a variety of cell types, including unwanted cell types. This is a particular problem when the generation and growth of neuronal axons (e.g., ascending sensory axons and descending motor axons) and the inhibition of astrogliosis (e.g., astroglial cell growth and scar formation) is desired. There exists a need in the art for improved compositions and methods for delivery of bioactive reagents (e.g., the promote the generation and growth of neuronal axons (e.g., ascending sensory axons and descending motor axons) and that concurrently inhibit astroglial cell growth and/or scar formation.

### SUMMARY OF THE INVENTION

The present invention relates to methods and compositions for altering (e.g., augmenting or stimulating) differentiation and growth of cells (e.g., neural progenitor cells and neurons). In particular, the present invention relates to compositions comprising one or more self-assembling peptide amphiphiles (e.g., in solution or that generate (e.g., self-assemble into) nanofibers (e.g., that are able to encapsulate cells and promote cellular differentiation (e.g., neurite development))) and methods of using the same. Compositions and methods of the present invention find use in research, clinical (e.g., therapeutic) and diagnostic settings.

Accordingly, in some embodiments, the present invention provides a method of altering development of a neuron comprising contacting the neuron with a composition comprising a peptide amphiphile. In some embodiments, altering development of a neuron comprises axonal growth. In some embodiments, the axonal growth comprises descending motor fiber growth. In some embodiments, the axonal growth comprises ascending sensory fiber growth. In some embodiments, altering development occurs through a lesion site. In some embodiments, altering development of a neuron is accompanied by reduced astrogliosis. In some embodiments, the peptide amphiphile comprises an IKVAV sequence and/or other laminin epitope. In some embodiments, the neuron is a neuron in a spinal cord that has been damaged. In some embodiments, the spinal cord has been damaged by traumatic spinal cord injury. In some embodiments, the neuron is a sensory neuron. In some embodiments, the neuron is a motor neuron. In some embodiments, altering development of a neuron comprises promoting development of the neuron. In some embodiments, altering development of a neuron comprises regenerating development of a damaged neuron. In some embodiments, the composition comprising a peptide amphiphile further comprises a growth factor. In some embodiments, the growth factor comprises a neurotrophic factor. The present invention is not limited to a particular neurotrophic factor. Indeed, a variety of neurotrophic factors are contemplated to be useful in the present invention including, but not limited to, Nerve growth factor (NGF), Brain-derived neurotrophic factor (BDNF), Neurotrophin-3 (NT-3), Neurotrophin-4/5 (NT-4/5), Ciliary neurotrophic factor (CNTF), Leukemia inhib. factor=chol. neuronal diff. factor (LIF/CDF), Cardiotrophin-1, Basic fibroblast growth factor (bFGF), Acidic fibroblast growth factor (aFGF), Fibroblast growth factor-5 (FGF-5), Insulin, Insulin-like growth factor I (IGF-I), Insulin-like growth factor Ii (IGF-II), Transforming growth factor β1 (TGFβ1), Transforming growth factor β2 (TGFβ2), Transforming growth factor β3 (TGFβ3), Activin, Glial cell-derived neurotrophic factor (GDNF), MidkineHeparin-binding neurotrophic factor (HBNF), Pleiotrophin, Epidermal growth factor (EGF), Transforming growth factor α (TGFα), Schwannoma-derived growth factor, Heregulin (neuregulin, ARIA), Interleukin 1, Interleukin 2, Interleukin 3, Interleukin 6, Axon ligand-1 (Al-1), elf-1, ehk1-L, and LERK2. In some embodiments, the neuron is a neurite.

The present invention also provides a method for treating a subject comprising: providing a subject with a damaged nerve, and administering a composition comprising a peptide amphiphile to the subject under conditions such that neuron growth occurs in the subject. In some embodiments, the neuron growth comprises axonal growth. In some embodiments, the axonal growth comprises descending motor fiber growth. In some embodiments, the axonal growth comprises ascending sensory fiber growth. In some embodiments, the neuron growth comprises axonal growth at the site of the damaged nerve. In some embodiments, the neuron growth is accompanied by reduced astrogliosis in the subject. In some embodiments, the neuron growth is accompanied by reduced scar formation in the subject. In preferred embodiments, the reduced astrogliosis and the reduced scar formation occur at the site of nerve damage. In some embodiments, the damaged nerve is a nerve in a spinal cord that has been damaged. In some embodiments, the damaged nerve has been damaged by traumatic spinal cord injury. In some embodiments, the damaged nerve comprises a damaged sensory neuron. In some embodiments, the damaged nerve comprises a damaged motor neuron. In some embodiments, neuron growth comprises regenerating development of a damaged neuron. In some embodiments, the composition comprising a peptide amphiphile further comprises a growth factor. In some embodiments, the growth factor comprises a neurotrophic factor. In some embodiments, administering comprises parenteral administration of an aqueous solution of the peptide amphiphile. In some embodiments, the peptide amphiphile forms a nanofiber gel upon contact with the damaged nerve. In some embodiments, the peptide amphiphile comprises a fluorescent agent. In some embodiments, the fluorescent agent comprises a pyrenebutyl moiety. In some embodiments, the composition comprising a peptide amphiphile is co-administered with one or more other agents. In some embodiments, the one or more other agents are selected from the group consisting of a neurotrophic factor, an inhibitor of a neuronal growth inhibitor, a neuronal growth attractant and a neuronal growth inhibitor. In some embodiments, the inhibitor of a neuronal growth inhibitor inhibits the expression and/or activity of Nogo, Ryk, Ryk-like inhibitors, sFRP, sFRP-like substances, MAG, Omgp, Wnt or CSPG.

The present invention also provides a pharmaceutical composition comprising a peptide amphiphile comprising an IKVAV sequence and/or other laminin epitope. In some embodiments, the composition is configured to alter neuron growth in a subject. In some embodiments, altering neuron growth comprises promoting neuron growth. In some embodiments, the peptide amphiphile comprises a SLSL sequence. In some embodiments, the SLSL sequence provides self-assembly of the peptide amphiphile that is therapeutically useful. In some embodiments, the peptide amphiphile comprises an A3 sequence. In some embodiments, the A3 sequence provides self-assembly of the peptide amphiphile that is therapeutically useful. In some embodiments, the peptide amphiphile comprises a heteroatom. Multiple heteroatoms are contemplated to be useful in the present invention (e.g., to distinguish one peptide amphiphile from another) including, but not limited to, a Br, I or F heteroatom. In some embodiments, the peptide amphiphile comprises a branching group. In some embodiments, the branching group improves the availability of a peptide epitope present within the peptide amphiphile. In some embodiments, the branching group comprises a modified lysine residue at its N-terminus.

In some embodiments, the present invention provides neural progenitor cells encapsulated within nanofiber scaffolds comprising peptide-amphipile (PA) compositions. The encapsulated neural progenitor cells find particular use in the presentation of bioactive peptide to the cells at high density. In some embodiments, the compositions and methods of the present invention find use in the delivery of bioactive peptides that induce neural progenitor cell differentiation.

Accordingly, in some embodiments, the present invention provides a system, comprising: a plurality of neural progenitor cells; and a nanofiber structure, wherein the nanofiber structure comprises peptide-amphiphiles, and wherein the plurality of neural progenitor cells are encapsulated inside the nanofiber structure. In certain embodiments, the peptide is a bioactive peptide (e.g., that induces neural progenitor cell differentiation or development). In some embodiments, the bioactive peptide is a growth factor, a hormone, or a differentiation factor. In some embodiments, the bioactive peptide comprises a bioactive epitope (e.g., IKVAV or other laminin epitope).

The present invention further provides a method, comprising: providing a plurality of neural progenitor cells; and a plurality of peptide-amphiphiles; and delivering the peptide-amphiphiles to the neural progenitor cells under conditions such that the neural progenitor cells are encapsulated inside a nanofiber structure, wherein the nanofiber structure comprises the peptide-amphiphiles. In some embodiments, the peptide is a bioactive reagent (e.g., a hormone, growth factor, or differentiation factor). In preferred embodiments, the encapsulation of the neural progenitor cells results in delivery of the peptide at a high local concentration. In some embodiments, the encapsulation results in selective differentiation of the cells. In some embodiments, the bioactive peptide comprises a bioactive epitope (e.g., IKVAV or other laminin epitope).

The present invention additionally provides a kit, comprising a plurality of neural progenitor cells; and a plurality of peptide-amphiphiles. In certain embodiments, the peptide is a bioactive peptide (e.g., that induces cell differentiation or development). In some embodiments, the bioactive peptide is a growth factor, a hormone, or a differentiation factor. In some embodiments, the bioactive peptide comprises a bioactive epitope (e.g., IKVAV).

The present invention also provides a kit, comprising a peptide amphiphile and a neurotrophic agent.

### DESCRIPTION OF THE FIGURES

FIG. 1(A) shows a molecular graphics illustration of an IKVAV-containing peptide amphiphile molecule and its self-assembly into nano-fibers. FIG. 1(B) shows a scanning electron micrograph of an IKVAV nanofiber network formed by adding cell media (DMEM) to a peptide amphiphile aqueous solution. FIGS. 1(C and D) show micrographs of the gel formed by adding to IKVAV peptide amphiphile solutions to (C) cell culture media and (D) cerebral spinal fluid. FIG. 1(E) shows a micrograph of an IKVAV nanofiber gel surgically extracted from an enucleated rat eye after intraocular injection of the peptide amphiphile solution.

FIG. 2 shows cell survival and morphology of NPCs encapsulated in IKVAV-PA gels or cultured on poly-(D-lysine) (PDL)-coated cover slips. Cell survival was determined at (A) 1 day, (B), 7 days, and (C) 22 days in vitro. FIG. 2(D) shows quantification of cell survival expressed as a percentage of total cells. FIG. 2(E) shows that cell body areas of differentiated neurons in the IKVAV-PA gels were significantly larger than those of controls at both 1 and 7 days (*P<0.05, **P<0.01). FIG. 2(F) shows TEM of NPC encapsulated in an IKVAV-PA gel at 7 days.

FIG. 3 shows a quantification of cell migration within a nanofiber network. FIG. 3(A) shows quantification of the migration of NPCs from three representative neurospheres encapsulated in an IKVAV-PA gel. FIG. 3(B) shows the three neurospheres for which the data in (A) were collected at 1 day (top) and 14 days (bottom) in vitro. FIG. 3(C) shows a brightfield image of an encapsulated NPC neurosphere in an IKVAV-PA gel less than 24 hours after plating.

FIGS. 4 (A and B) show NPCs cultured under different experimental conditions. FIG. 4(C) shows immunocytochemistry of an NPC neurosphere encapsulated in an IKVAV-PA nanofiber network at 7 days. FIG. 4(D) shows NPCs cultured on laminin-coated cover slips at 1 day. FIG. 4(E) shows NPCs cultured on laminin-coated cover slips at 7 days. FIG. 4(F) shows the percentage of total cells that differentiated into neurons (β-tubulin). FIG. 4(G) shows the percentage of total cells that differentiated into astrocytes (GFAP+). FIG. 4H shows the percentage of total cells that differentiated into neurons after 1 day in nanofiber networks containing different amounts of IKVAV-PA and EQS-PA (solid line) and in EQS-PA nanofiber networks to which different amounts of soluble IKVAV peptide were added (dashed line).

FIG. 5 shows the percentage of total cells that differentiated into neurons in a two-dimensional culture on substrates coated with IKVAV-PA nanofibers and substrates coated with IKVAV peptide.

FIG. 6 shows the structure and characterization of PA1 and PA2. (a) Chemical structure of several PAs of the present invention. The peptide sequence is terminated at the N-terminus with a palmityl tail (PA1) or a pyrenebutyl tail in a fluorescent version (PA2). This peptide sequence is modified from that described elsewhere (See, e.g., Nomizu, M. et al., FEBS Lett 365, 227-31 (1995)) and was used due to its slower kinetics of gelation. (b) MALDI-TOF mass spectra of PA1 (left) and PA2 (right). The peaks at −30 and −60 from the parent peak in both cases are due to the loss of CH2═OH(+) from the serine side chains. (c) 1H-NMR spectra of PA1 (left) and PA2 (right), taken in dTFA to minimize aggregation. The region from ppm0-6 is magnified for visibility. The region above 6 is empty except for aromatic peaks due to the pyrenyl protons, which appear in the spectrum of PA1, and small broad peaks from exchangeable amide protons in both spectra.

FIG. 7 shows the characterization of the nanofiber gel using atomic force microscopy and rheological data. FIG. 7(a) shows atomic force microscopy image showing the slower gelling PA1 (Palmityl-IKVAV-PA fibers) in gelled state. The individual fibers, about 7 nm in width, can be seen in this image along with a partial fiber network. FIGS. 7(b) through (d) present rheological data for PA1 and PA2 (Pyrene-IKVAV-PA or fluorescent IKVAV-PA gel). The data shown are taken at 4.22 Hz and 3% strain, which falls in the linear viscoelastic regime for all three PAs. Error bars are 1 standard deviation. FIG. 7(b) shows the complex modulus. PA1 and PA2 varieties are comparable in stiffness. FIG. 7(c) shows complex viscosity. FIG. 7(d) shows damping factors (G″/G′) for the two PAs. Both materials gelled (G″/G′<0.2) with the addition of DMEM.

FIG. 8 shows the IKVAV gel decreases astrocyte lineage commitment by postnatal neural progenitor cells in vitro. Fewer astrocytes (GFAP+ cells) are generated from postnatal neural progenitor cells encapsulated in IKVAV gel versus cells cultured on poly-D-lysine (PDL)/laminin-coated coverslips.*p<0.01 by t test.

FIG. 9 shows IKVAV peptide amphiphile (PA) solution self-assembles in vivo and diminishes glial scar formation. FIG. 9(a) shows a schematic representation showing individual PA molecules assembled into a bundle of nanofibers interwoven to produce the gel. FIG. 9(b) shows a scanning electron micrograph showing the network of nanofibers. Scale bar: 200 nm. FIG. 9(c) shows a longitudinal section showing the fluorescent IKVAV gel in the injured spinal cord 24 hours after injection. FIG. 9(d) shows a longitudinal section of spinal cord showing the gel 5 weeks around the injection track (arrowheads), but also at a distance from the injection site (long vertical arrows in fluorescence image) (Scale bars in c and d: 200 μm). FIGS. 9 (e) and (f) show IKVAV gel attenuates astrogliosis in vivo following spinal cord injury. FIG. 9(e) shows GFAP immunofluorescence is reduced in the lesion site of the gel-treated animal compared to the control. Scale bar: 20 μm. FIG. 9(f) shows the control and gel-injected groups do not differ at 4 days, but at 77 days GFAP immunofluorescence levels in the gel-injected spinal cord are significantly reduced compared to the control (*p<0.04 by t test).

FIG. 10 shows spinal cord injury results in an early hypertrophic but later hyperplastic glial response. Representative confocal Z-stacks of sections immunostained for GFAP showing uninjured spinal cord as well as the lesion site 4 days and 11 weeks after spinal cord injury. Note the hypertrophic morphology of the reactive astrocytes at 4 days post injury (arrowheads) and the obvious increase in number of GFAP+ cells at 11 weeks post injury. Scale bar: 20 μm.

FIG. 11 shows that the IKVAV gel promotes regeneration of motor axons following spinal cord injury. Representative Neurolucida tracings of BDA-labelled descending motor fibers within a distance of 500 μm rostral to the lesion in vehicle-injected and gel-injected animals. The dotted grey lines demarcate the borders of the lesion. The bar graphs show the extent to which labelled corticospinal axons penetrated the lesion. By 11 weeks 50% of the axons in the gel-injected group (black bars) extended half of the way into the lesion and 40% of the axons in the gel-injected group grew beyond the lesion into the caudal spinal cord. By contrast, no axons were ever seen crossing even 25% of the way into the lesion in the vehicle-injected animals (red bars). *The groups (representing tracing of 130 individual axons) differ from each other at p<0.03 by the Wilcoxon rank test. In all sections shown, rostral is to the top and dorsal is to the left. All scale bars: 100 μm

FIG. 12 shows that the IKVAV gel promotes regeneration of sensory axons following spinal cord injury. Representative Neurolucida tracings of BDA-labeled ascending sensory fibers within a distance of 500 μm caudal to the lesion in vehicle-injected and gel-injected animals. The dotted grey lines demarcate the borders of the lesion. Bar graph showing the extent to which labelled axons entered and grew through the lesion. At 2 weeks post injury only a few fibers entered the lesion and no fibers in either group penetrated as far as 25% of the way across the lesion. By 11 weeks approximately 60% of labelled axons in the gel-injected animals (black bars) entered the lesion compared to only about 20% of the fibers in control animals (red bars). At 11 weeks, significantly more axons penetrated the lesion in the gel-injected animals (black bars) than in the vehicle-injected group (red bars), and only in the gel-injected group did fibers grow 50% into the lesion or beyond. **The groups differ from each other at p<0.05 by the Wilcoxon rank test.

FIG. 13 shows that the IKVAV gel promotes functional recovery as analyzed by the BBB open field locomotor scale. FIG. 13(a) shows a graph showing mean mouse BBB locomotor scores. The groups differ from each other at p<0.04 by ANOVA with repeated measures. * Tukey's HSD post hoc t tests showed that scores differed at p<0.045 at every time point 5 weeks after the injury and thereafter. FIG. 13(b) shows a graph showing mean rat BBB locomotor scores. ANOVA with repeated measures showed that the groups differed from each other (p<0.03). Tukey's HSD post hoc t tests showed no difference between sham and vehicle-treated animals, however, the gel inject group differed from the other groups at 5 weeks (*p<0.03) and at all times thereafter (**p<0.02).

FIG. 14 shows peptide amphiphiles of the present invention.

FIG. 15 shows slow-gelling peptide amphiphiles of the present invention.

FIG. 16 shows various peptide amphiphiles comprising a heteroatom dopant of the present invention.

FIG. 17 shows peptide amphiles comprising branching groups of the present invention.

### DEFINITIONS

As used herein, the term “pluripotent” means the ability of a cell to differentiate into multiple different types of cells (e.g., terminally differentiated cells). For example, pluripotent cells include those that can differentiate into the three main germ layers: endoderm, ectoderm, and mesoderm.

As used herein, the term “progenitor cell” refers to a cell that is capable of differentiating into a specific cell type.

As used herein, the terms “transplant cells” and “graft material” refer broadly to the component (e.g., tissue or cells) being grafted, implanted or transplanted. As used herein, the term “transplantation” refers to the transfer or grafting of tissues or cells from one part of a subject to another part of the same subject, or to another subject, or the introduction of biocompatible materials into or onto the body. As used herein, in some embodiments, a transplanted tissue may comprise a collection of cells of identical or similar composition, or derived from an organism (e.g., a donor), or from an in vitro culture (e.g., a tissue culture system).

The term “recipient of transplanted cells” as used herein, refers broadly to a subject undergoing transplantation and receiving transplanted cells.

As used herein, the term “cell culture” refers to any in vitro culture of cells, including but not limited to continuous cell lines (e.g., with an immortal phenotype), primary cell cultures, and finite cell lines (e.g., non-transformed cells).

The term “in vitro” refers to an artificial environment and to processes or reactions that occur within an artificial environment. The term “in vivo” refers to the natural environment (e.g., an animal or a cell) and to processes or reactions that occur within a natural environment. The definition of an in vitro versus in vivo system is particular for the system under study. As used herein, an in vitro system refers to studies of cells or processes in an artificial environment, such as in tissue culture vessels and apparatus, whereas study of the same system in an in vivo context refers to the study of cells or processes within an organism, such as a rat, mouse, or human.

As used herein, the term “primary cell” or “primary culture” refers to a cell or a culture of cells that have been explanted directly from an organism, organ, or tissue. Primary cultures are typically neither transformed nor immortal.

The term “tissue culture” as used herein, refers to a collection of techniques for the growth and maintenance of cells in the laboratory. Such techniques may involve tissue culture dishes or other vessels, incubators and sterility containment devices, as known in the art.

As used herein, the term “exogenous” is used interchangeably with the term “heterologous” to refer to a substance coming from some source other than its native source. For example, the terms “exogenous protein,” or “exogenous cell” refer to a protein or cell from a non-native source or location, and that have been artificially supplied to a biological system. In contrast, the terms “endogenous protein,” or “endogenous cell” refer to a protein or cell that are native to the biological system, species or individual.

As used herein, the term “stem cells” refers to cells that can self-renew and differentiate into multiple lineages. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

The terms “embryonic stem cell” (“ES cell”) refer to cells derived from mammalian blastocysts, which are self-renewing and have the ability to yield many or all of the cell types present in a mature animal. Human embryonic stem cell lines suitable for use with the methods and compositions of the present invention include but are not limited to those produced by the following institutions: BresaGen, Inc., Athens, Ga.; CyThera, Inc., San Diego, Calif.; ES Cell International, Melbourne, Australia; Geron Corporation, Menlo Park, Calif.; Goteborg University, Goteborg, Sweden; Karolinska Institute, Stockholm, Sweden; Maria Biotech Co. Ltd.—Maria Infertility Hospital Medical Institute, Seoul, Korea; MizMedi Hospital—Seoul National University, Seoul, Korea; National Centre for Biological Sciences/Tata Institute of Fundamental Research, Bangalore, India; Pochon CHA University, Seoul, Korea; Reliance Life Sciences, Mumbai, India; Technion University, Haifa, Israel; University of California, San Francisco, Calif.; and Wisconsin Alumni Research Foundation, Madison, Wis. The human ES cells listed on the Human Embryonic Stem Cell Registry to be created by the National Institutes of Health find use in the methods and compositions of the present invention. However, human ES cells not listed on the NIH registry are also contemplated to find use in embodiments of the present invention (e.g., when it is desirable to prevent ES contamination with nonhuman-derived materials).

As used herein the term “feeder cells” refers to cells used as a growth support in a tissue culture system. In preferred embodiments, the term “feeder cells” refers to embryonic “striatum cells,” while in other embodiments the term “feeder cells” refers to stromal cells.

As used herein, the terms “peptide amphiphile” and “PA” and “amphiphile” refer to a composition that comprises an organic moiety comprising a hydrophobic region (e.g., a linear peptide chain (e.g., a palmitoyl group) or a hydrophobic ring structure (e.g., pyrenebutyl)) joined to a structural region (e.g., comprising sequences (e.g., β-sheets) that can alter and/or influence packing and self-assembly of the peptide amphiphile) joined to a functional region (e.g., comprising a peptide epitope (e.g., IKVAV and/or YIGSR sequence). The peptide moiety may comprise one or more other regions (e.g., charged amino acid or sequence thereof (e.g., adjacent to the hydrophobic region, structural region or functional region)) that can determine the charge of the peptide amphiphile. In addition to being described herein, peptide amphiphiles that find use in the present invention are described in U.S. Pat. Apps. 20050272662, 20050209145, 20050208589, 20040258726, 20040022718, 20040018961, 20040001893, and international applications WO/05056576, WO/05056039, WO/05003292, WO/04106359, WO/04072104, WO/04046167, WO/04018628, WO/04003561, WO/03090255, WO/03084980, WO/03070749, and WO/03054146, each of which is hereby incorporated by reference in its entirety.

As used herein, the term “isolated” when used in relation to material (e.g., a cell) refers to a material that is identified and separated from at least one component or contaminant with which it is ordinarily associated in its natural source. An isolated material is such present in a form or setting that is different from that in which it is found in nature.

As used herein, the term “purified” or “to purify” refers to the removal of components (e.g., contaminants) from a sample.

As used herein, the term “subject” refers to any animal (e.g., a mammal), including, but not limited to, humans, non-human primates, rodents, and the like (e.g., that is to be the recipient of a particular treatment (e.g., administration of an amphiphile of the present invention). The terms “subject” and “patient” are used interchangeably in reference to a human subject, unless indicated otherwise herein.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

The terms “test compound” and “candidate compound” refer to any chemical entity, pharmaceutical, drug, and the like that is a candidate for use to treat or prevent a disease, illness, injury (e.g., spinal cord injury), sickness, or disorder of bodily function (e.g., neurodegenerative disease). Candidate compounds comprise both known and potential therapeutic compounds (e.g., agents known to stimulate or inhibit neuron growth as well as those whose effect on neural cell growth are yet to be determined (e.g., using systems and methods of the present invention). A candidate compound can be determined to be therapeutic by screening using the screening methods of the present invention.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that-are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA can be quantified; other minor species of RNA which hybridize to the transgene probe are generally not considered in the quantification of the expression of the transgenic mRNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

The term “vector” refers to nucleic acid molecules that transfer DNA segment(s) from one cell to another. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non-transfected cells, or reporter proteins, by means of which an effect on expression or activity or function of the reporter protein can be monitored.

The term “expression cassette” refers to a chemically synthesized or recombinant DNA molecule containing a desired coding sequence and appropriate nucleic acid sequences necessary for the expression of the operably linked coding sequence either in vitro or in vivo. Expression in vitro includes expression in transcription systems and in transcription/ translation systems. Expression in vivo includes expression in a particular host cell and/or organism. Nucleic acid sequences necessary for expression in prokaryotic cell or in vitro expression system usually include a promoter, an operator (optional), and a ribosome binding site, often along with other sequences. Eukaryotic in vitro transcription systems and cells are known to utilize promoters, enhancers, and termination and polyadenylation signals. Nucleic acid sequences necessary for expression via bacterial RNA polymerases, referred to as a transcription template in the art, include a template DNA strand which has a polymerase promoter region followed by the complement of the RNA sequence desired. In order to create a transcription template, a complementary strand is annealed to the promoter portion of the template strand.

The term “expression vector” refers to a vector comprising one or more expression cassettes.

The term “siRNAs” refers to short interfering RNAs. In some embodiments, siRNAs comprise a duplex, or double-stranded region, of about 18-29 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures.

As used herein, the terms “effective amount” and “therapeutically effective amount” refer to an amount of a compound (e.g., peptide amphiphile or a solution comprising the same) sufficient to effect beneficial or desired results (e.g., to effect neuronal growth (e.g., using methods of the present invention)). An effective amount can be administered in one or more administrations, applications or dosages and is not intended to be limited to a particular formulation or administration route.

As used herein, the terms “administration” and “administering” refer to the act of giving a drug, prodrug, test compound or other agent, or therapeutic treatment (e.g., compositions of the present invention) to a cell or subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administering” refer to the administration of at least two agent(s) (e.g., a composition comprising a peptide amphiphile comprising one type of peptide epitope (e.g., IKVAV) and one or more other agents (e.g., a peptide amphiphile comprising a second type of peptide amphiphile (e.g., YIGSR))) or therapies to a cell or subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “neurite” refers to a neuron in the growth process. Thus, the terms “neurite growth” or “neurite development” refer to the extension of axonal processes from the neuron (e.g., cell body).

As used herein, the terms “contact” or “contacting” refer to any manner in which a composition of the present invention (e.g., a solution or nanofiber gel comprising a peptide amphiphile of the present invention) is brought into a position where it can mediate or alter (e.g., stimulate, augment, inhibit, etc.) growth of a neuron. For example, “contacting” may comprise injecting a solution comprising a PA into an area where there are neurons.

### DETAILED DESCRIPTION OF THE INVENTION

The central nervous system comprises the brain and the spinal cord. All other nerves in the body comprise the peripheral nervous system. Efferent nerves carry messages from the central nervous system to all parts of the body (the periphery) whereas afferent nerves carry information such as pain intensity from the periphery to the central nervous system. There are two types of efferent nerves: somatic, which go to skeletal muscles, and autonomic, which go to smooth muscles, glands and the heart. Messages in the form of electrical activity are conducted along the nerve fibers or axons. Between the terminus of the axon and the muscle or gland that the nerve controls (innervates), there is a gap called the synapse or synaptic cleft. When the conducted electrical impulse (action potential) reaches the nerve terminus, it provokes the release of chemicals called neurotransmitters. These chemicals diffuse across the synaptic cleft and react with a specialized structure (receptor) on the postjunctional membrane. The receptor is then said to be activated or excited, and its activation triggers a series of chemical events resulting ultimately in a biological response such as muscle contraction. The processes involving neurotransmitter release, diffusion and receptor activation are referred to collectively as transmission. There are many types of transmission, and they are named for the specific neurotransmitter involved. Thus, cholinergic transmission involves the release of the neurotransmitter, acetylcholine, and its activation of the postsynaptic receptor. Things that bind to and activate receptors are called agonists. Thus, acetylcholine is the endogenous agonist for all cholinergic receptors.

After leaving the central nervous system, somatic nerves to skeletal muscles have only one synapse, namely, that between the nerve terminus and the muscle it innervates. The neurotransmitter at that synapse is acetylcholine. Thus, this myo-(for muscle)-neural junction is one site of cholinergic transmission. The postjunctional receptor is called the motor end plate. Autonomic nerves, in contrast to somatic nerves, have an additional synapse between the central nervous system and the innervated structure (end organ). These synapses are in structures called ganglia, and these are nerve-to-nerve junctions instead of nerve-to-end organ junctions. Like somatic nerves, however, autonomic nerves also have a final nerve-to-end organ synapse. The neurotransmitter in autonomic ganglia is also acetylcholine; hence, this represents another site of cholinergic transmission. The motor end plate and the ganglionic receptors can also be activated by exogenously added nicotine. Thus, nicotine is an agonist for this particular subfamily of cholinergic receptors which are called nicotinic, cholinergic receptors.

There are two anatomically and functionally distinct divisions of the autonomic nervous system: the sympathetic division and the parasympathetic division. The preganglionic fibers of the two divisions are functionally identical, and they innervate nicotinic, cholinergic receptors in ganglia to initiate action potentials in the postganglionic fibers. Only the postganglionic fibers of the parasympathetic division, however, are cholinergic. The postganglionic fibers of the sympathetic division generally, but not always, secrete norepinephrine. The cholinergic receptors innervated by the postganglionic fibers of the parasympathetic division of the autonomic nervous system can also be activated by exogenously added muscarine, an agonist found in small amounts in the poisonous mushroom, Amanita muscaria. These constitute a second subset of cholinergic receptors which are called muscarinic, cholinergic receptors.

Although the receptors in ganglia and the motor end plate both respond to nicotine, they actually constitute two distinct subgroups of nicotinic receptors. Each of the three families of cholinergic receptors can be blocked by specific receptor antagonists to prevent their activation by endogenous acetylcholine or added agonists. Thus, specific blockers are known for cholinergic, muscarinic receptors innervated by postganglionic fibers of the parasympathetic division of the autonomic nervous system, for cholinergic, nicotinic receptors in both sympathetic and parasympathetic ganglia, and for cholinergic nicotinic receptors at the myoneural junction (motor end plates) of the somatic nervous system. When these receptors are blocked, the on-going biological activity associated with their normal and continuous activation is lost. For example, blockade of the motor end plate leads to generalized, flaccid paralysis.

There are some anomalous fibers in the sympathetic division of the autonomic nervous system. For example, the sympathetic postganglionic nerves that go to sweat glands are cholinergic instead of adrenergic, like most other sympathetic fibers, and they innervate mucarinic receptors. The sympathetic nerve to the adrenal gland innervates a receptor that is nicotinic like all autonomic ganglia, but there is no postganglionic fiber. The gland itself is analogous to a postganglionic sympathetic fiber, but, instead of secreting a neurotransmitter, it secretes epinephrine and norepinephrine into the blood stream, where they function as hormones. These hormones activate adrenergic receptors throughout the body.

The spinal cord conducts sensory information from the peripheral nervous system (e.g., both somatic and autonomic) to the brain, and it also conducts motor information from the brain to various effectors (e.g., skeletal muscles, cardiac muscle, smooth muscle, or glands). The spinal cord also serves as a minor reflex center.

The brain receives sensory input from the spinal cord as well as from its own (e.g., cranial) nerves (e.g., trigeminal, vestibulocochlear nerve, olfactory and optic nerves) and devotes most of its volume and computational power to processing its various sensory inputs and initiating appropriate and coordinated motor outputs. Both the spinal cord and the brain comprise white matter (e.g., bundles of axons each coated with a sheath of myelin) and gray matter (e.g., masses of cell bodies and dendrites each covered with synapses).

Both the spinal cord and brain are covered in three continuous sheets of connective tissue known as the meninges. From outside in, these are the dura mater pressed against the bony surface of the interior of the vertebrae and the cranium; the arachnoid; and the pia mater. The region between the arachnoid and pia mater is filled with cerebrospinal fluid (CSF).

This CSF of the central nervous system is unique. Cells of the central nervous system are bathed in CSF that differs from fluid serving as the ECF of the cells in the rest of the body. The fluid that leaves the capillaries in the brain contains far less protein than “normal” because of the blood-brain barrier, a system of tight junctions between the endothelial cells of the capillaries. This barrier creates problems in medicine as it prevents many therapeutic drugs from reaching the brain. The cerebrospinal fluid (CSF) is a secretion of the choroid plexus. CSF flows uninterrupted throughout the central nervous system through the central cerebrospinal canal of the spinal cord and through an interconnected system of four ventricles in the brain. CSF returns to the blood through veins draining the brain.

The Spinal Cord comprises 31 pairs of spinal nerves that align the spinal cord. These are “mixed” nerves as each contain both sensory and motor axons. However, within the spinal column, sensory axons pass into the dorsal root ganglion where their cell bodies are located and then on into the spinal cord itself, whereas motor axons pass into the ventral roots before uniting with the sensory axons to form the mixed nerves.

The spinal cord carries out two main functions. It connects a large part of the peripheral nervous system to the brain. Information (e.g., nerve impulses) reaching the spinal cord through sensory neurons are transmitted up into the brain. Signals arising in the motor areas of the brain travel back down the cord and leave in the motor neurons. The spinal cord also acts as a minor coordinating center responsible for some simple reflexes like the withdrawal reflex. Some of the cranial nerves (e.g., the optic and olfactory nerves) contain sensory axons only whereas some of the cranial nerves (e.g., the oculomotor nerve (e.g., that controls eyeball muscles)), contain motor axons only.

Signals cross over the spinal tracts. For example, impulses reaching the spinal cord from the left side of the body eventually pass over to tracts running up to the right side of the brain and vice versa. In some cases this crossing over occurs as soon as the impulses enter the cord. In other cases, it does not take place until the tracts enter the brain itself.

The cranial nerves emanate from the nervous tissue of the brain. In order to reach their targets they ultimately exit/enter the cranium through openings in the skull. Hence, their name is derived from their association with the cranium. The function of the cranial nerves is similar to the spinal nerves, the nerves that are associated with the spinal cord. The motor components of the cranial nerves are derived from cells that are located in the brain. These cells send their axons (e.g., bundles of axons outside the brain, the bundles themselves comprising the nerve) out of the cranium where they ultimately control muscle (e.g., eye movements, diaphragm muscles, muscles used for posture, etc.), glandular tissue (e.g., salivary glands), or specialized muscle (e.g., heart or stomach).

The sensory components of cranial nerves originate from collections of cells that are located outside the brain. These collections of nerve cell bodies are called sensory ganglia. They are similar functionally and anatomically to the dorsal root ganglia which are associated with the spinal cord. In general, sensory ganglia of the cranial nerves send out a branch that divides into two branches: a branch that enters the brain and one that is connected to a sensory organ. Examples of sensory organs are pressure or pain sensors in the skin and more specialized ones such as taste receptors of the tongue. Electrical impulses are transmitted from the sensory organ through the ganglia and into the brain via the sensory branch that enter the brain. In summary, the motor components of cranial nerves transmit nerve impulses from the brain to target tissue outside of the brain. Sensory components transmit nerve impulses from sensory organs to the brain.

Thus, the CNS is connected by ascending sensory pathways (e.g., somatosensory pathways ascending to the brain centers) and descending motor or regulatory pathways (e.g., controlling body movement descending from the brain to the spinal cord).

Unlike the peripheral nervous system, damage to central nervous system axons (e.g., spinal cord axons) have heretofore not been repairable, leading to permanent impairment of neural function (e.g., paralysis).

Spinal cord injury refers generally to any injury of the neurons within the spinal canal. Spinal cord injury can occur from a variety of events (e.g., trauma or disease to the vertebral column or the spinal cord itself). Most spinal cord injuries are the result of trauma to the vertebral column causing a fracture of the bone, or tearing of the ligaments with displacement of the bony column producing a pinching of the spinal cord. The majority of broken necks and broken backs, or vertebral fractures, do not cause any spinal cord damage; however, in 10-14% of the cases where a vertebral trauma has occurred, the damage is of such severity it results in damage to the spinal cord.

Patients with a spinal cord injury often are diagnosed as having tetraplegia (preferred to quadriplegia) or paraplegia. Tetraplegia refers to injuries to the cervical spinal cord and paraplegia refers to injuries below the cervical spinal cord. Patients with tetraplegia are slightly more common than patients with paraplegia.

It is estimated that the annual incidence of spinal cord injury (SCI), not including those who die at the scene of the accident, is approximately 40 cases per million population in the U.S., or approximately 11,000 new cases each year. The number of people in the U.S. who are alive today and who have SCI has been estimated to be between 721 and 906 per million population. This corresponds to between 183,000 and 230,000 persons.

Treatment options for patients with spinal cord injuries are limited. Various approaches have been utilized to treat SCI with limited success (See, e.g., Richardson et al., Nature 284, 264-265 (1980); Bradbury et al., Nature 416, 636-40 (2002); Schnell and Schwab, Nature 343, 269-72 (1990); GrandPre and Strittmatter, Nature 417, 547-51 (2002); Liu et al., J Neurosci 19, 4370-87 (1999); Lu et al., J Neurosci 24, 6402-9 (2004); Qiu et al., Neuron 34, 895-903 (2002); Nikulina et al., Proc Natl Acad Sci U S A 101, 8786-90 (2004); Pearse et al., Nat Med 10, 610-6 (2004); McDonald et al., Nat Med 5, 1410-2 (1999); Shaw et al., J Craniofac Surg 14, 308-16 (2003); Teng et al., Proc Natl Acad Sci U S A 99, 3024-9 (2002)). Often, patients with SCI are left with severe, permanent disabilities. Treatment of spinal cord injury and other injuries or diseases that result in neural cell damage have been limited due to the inability of existing therapies and treatments to regenerate ascending (e.g., somatosensory) as well as descending (e.g., motor) axonal fibers. Furthermore, molecular mechanisms that guide axons along the anterior-posterior (A-P) axis of the spinal cord are unknown.

Axonal connections are patterned along the A-P and dorsal-ventral (D-V) neuraxes, wiring a large number of neurons into an intricate network. Axon guidance along the D-V axis has been a major focus of study in a number of experimental systems in recent years. Four classes of axon guidance molecules have been described (See, e.g., Tessier-Lavigne and Goodman, 1996): long-range attractants, long-range repellents, contact-mediated attractants and contact-mediated repellents.

The dorsal spinal cord commissural neurons form several ascending somatosensory pathways, such as the spinothalamic tracts, that send pain and temperature sensations to the brain. The cell bodies of commissural neurons are located in the dorsal spinal cord. During embryonic development, commissural neurons project axons to the ventral midline. Once they reach the floor plate, they cross the midline and enter the contralateral side of the spinal cord. After midline crossing, commissural axons make a remarkably sharp anterior turn towards the brain. All dorsal spinal cord commissural axons along the entire anterior-posterior length of the spinal cord project anteriorly after midline crossing. The initial ventral growth of the commissural axons is controlled by a gradient of a diffusible chemoattractant, Netrin-1 (See, e.g., Serafini et al., 1994; Kennedy et al., 1994; Serafini et al., 1996). As the axons cross the midline, they lose responsiveness to Netrin-1 (See, e.g., Shirasaki et al., 1998). Interestingly, while losing responsiveness to Netrin-1 during midline crossing, commissural axons gain responsiveness to several chemorepellents, which are located in the midline and the ventral spinal cord (See, e.g., Zou et al., 2000). These repellents help to expel the axons from the midline and to turn axons from their dorsal-ventral trajectory into their longitudinal pathways along the anterior-posterior axis by preventing axons from overshooting into the contralateral ventral spinal cord and re-crossing the floor plate; the axons thus become “squeezed” into their longitudinal pathway (See, e.g., Zou et al., 2000).

New compositions and methods are needed for altering (e.g., promoting or inhibiting) neuronal growth and regeneration (e.g., following SCI or other forms of injury) while concurrently inhibiting astrogliosis (e.g., astroglial proliferation and scar formation). Novel compositions and methods for neuronal growth and regeneration could also be applied in the treatment of patients with other disorders involving neuronal dysfunction, such as neurodegenerative diseases. Specifically, compositions and methods are needed that are able to promote ascending and descending axonal growth (e.g., following injury to the spinal cord) that may be used therapeutically (e.g., to prevent paralysis in a subject following injury or disease).

Accordingly, the present invention provides methods and compositions for altering (e.g., augmenting or stimulating) differentiation and growth of cells (e.g., neural progenitor cells and neurons). In particular, the present invention relates to compositions comprising one or more self-assembling peptide amphiphiles (e.g., in solution or that generate (e.g., self-assemble into) nanofibers (e.g., that are able to encapsulate cells and promote cellular differentiation (e.g., neurite growth))) and methods of using the same. Compositions and methods of the present invention find use in research, clinical (e.g., therapeutic) and diagnostic settings.

Molecular recognition among ligands and receptors in biology requires appropriate presentation of epitopes. Peptide epitopes (e.g., adhesion ligands) play important roles in cell adhesion, attachment and stimulation of cellular signaling pathaways (e.g., pathways that result in cell proliferation, differentiation and maintenance of regular metabolic activities). Recently, there has been great interest in designing scaffolds that mimic cellular structures with artificial epitopes in order to trigger biological events (e.g., for use in regenerative medicine or targeted chemotherapy). Differences in cellular response have been reported with changes in distribution and structural presentation of the signals on these artificial cell scaffolds. For, example, varying the nanoscale separation between cell adhesion ligands has been found to improve the recognition of signals and subsequent proliferation of the cells. Among the various methodologies used to synthesize biomaterials, self-assembly is a particularly attractive tool to create scaffolds from solutions of molecules that can encapsulate cells and assemble in situ or in vivo.

Artificial three-dimensional (3D) scaffolds that store or attract cells, and then direct cell proliferation and differentiation, find use in regenerative medicine, drug screening, and research uses. Earlier work demonstrated that tissue regeneration using cell-seeded artificial scaffolds is possible, either by implanting the scaffolds in vivo or maintaining them in a bioreactor followed by transplantation (See, e.g., Langer and Vacanti, Science 260, 920 (1993); Lendlein, R. Langer, Science 296, 1673 (2002); Teng et al., Proc. Natl. Acad. Sci. U.S.A. 99, 3024 (2002); Lu et al., Biomaterials 21, 1837 (2000); Niklason., Science 284, 489 (1999); Nehrer et al., J. Biomed. Mater. Res. 38, 95 (1997); Atala et al., J. Urol. 150, 745 (1993); Wald et al., Biomaterials 14, 270 (1993); Yannas, Science 215, 174(1982)). The scaffold materials used in most previous work have been biodegradable, nonbioactive polymers such as poly(L-lactic acid) and poly(glycolic acid) (See, e.g., Mooney et al., Biomaterials 17, 1417 (1996); Mikos et al., Biomaterials 15, 55 (1994)), as well as biopolymers such as collagen, fibrin, and alginate (See, e.g., Lavik et al., Methods Mol. Biol. 198, 89 (2002); Hsu et al., Invest. Ophthalmol. Vis. Sci. 41, 2404 (2000); Chamberlain et al., J. Neurosci. Res. 60, 666 (2000); Butler et al., Br. J. Plast. Surg. 52, 127 (1999); Orgill et al., Plast. Reconstr. Surg. 102, 4 23 (1998); Chang et al., J. Biomed. Mater. Res. 55, 503 (2001); Atala et al., J. Urol. 150, 745 (1993)). The polymer scaffolds are typically prefabricated porous objects, fabrics, or films that are seeded with cells of the tissue to be regenerated. In the case of biopolymers, a common form of the scaffold is an amorphous gel in which cells can be encapsulated (See, e.g., Lim and Sun, Science 210, 908 (1980); Hortelano et al., Blood 87, 5095 (1996); Xu and Liu, FASEB J. 16, 213 (2002)).

Experiments conducted during the course of development of the present invention demonstrated the formation of solid scaffolds (e.g., in vivo) that incorporate peptide sequences known to direct cell differentiation and to form by self-assembly from aqueous solutions of peptide amphiphiles. In some embodiments, the scaffolds comprise nanofiber networks formed by the aggregation of the amphiphilic molecules (e.g., triggered by the addition of neural progenitor cell suspensions to the aqueous solutions or by exposure to cerebral spinal fluid). The nanofibers can be customized through the peptide sequence for a specific cell response, and the scaffolds formed by these systems can be delivered to living cells and/or tissues by simply injecting a liquid (e.g., peptide amphiphile solutions). Experiments further demonstrated that an artificial scaffold can direct the differentiation of neural progenitor cells into neurons (See, e.g., Examples 1-8) while suppressing astrocyte differentiation, and furthermore, that administration (e.g., injection into an injured spinal cord) of a composition comprising a peptide amphiphile of the present invention to a subject with an injured spinal cord reduces astrogliosis at the site of injury, promotes substantial regeneration of sensory and motor fibers, and significantly enhances behavioral recovery (e.g., mobility of limbs paralyzed prior to such treatment (See, e.g., Examples 9-13).

Accordingly, in some embodiments, the present invention provides a composition comprising a peptide amphiphile (PA) for delivering and/or presentation of a peptide epitope to a target (e.g., a neural progenitor cell, a neuron or other cellular target). In some preferred embodiments, delivery and/or presentation of a peptide epitope promotes neuron growth (e.g., neurite growth (e.g., generation of descending (e.g., motor) and/or ascending (e.g., sensory) fibers (e.g., through a lesion)) and/or proliferation. In other preferred embodiments, the present invention provides a method of altering (e.g., promoting, facilitating or stimulating) neuron growth comprising providing a neuron (e.g., in vivo, ex vivo, or in vitro) and administering to the neuron a composition comprising a PA of the present invention. In some preferred embodiments, the composition comprising a PA forms a nanofiber gel when in contact with a neuron. In some embodiments, the neuron is a neuron within a spinal cord (e.g., a damaged spinal cord (e.g., a spinal cord damaged by a traumatic spinal cord injury)). In some embodiments, the neuron is a sensory neuron. In some embodiments, the neuron is a motor neuron. In some embodiments, the composition comprising a PA inhibits astroglial cell growth and scar formation while concurrently stimulating neuronal (e.g., motor or sensory fiber) growth. In some embodiments, administrating a composition comprising a PA of the present invention to a subject results in a behavioral improvement in the subject (e.g., the subject is able to move a limb (e.g., a leg or arm) paralyzed prior to treatment). In some embodiments, the composition comprising a PA comprises one or more other agents (e.g., a growth factor (e.g., a neurotrophic factor) or an inhibitor of an inhibitor of axonal growth).

Exemplary methods and compositions of the present invention are described in greater detail below. However, the present invention is not limited to the compositions and methods described herein. One skilled in the art understands that additional compositions and uses are within the scope of the present invention.

I. Peptide-Amphiphile Compositions

The peptide-amphiphile (PA) compositions used in the present invention can be synthesized using preparatory techniques well-known to those skilled in the art—preferably, by standard solid phase chemistry, with alkylation or other modification of the N-terminus of the peptide component with a hydrophobic moiety, mono or di-alkyl moieties attached to the N- or C-termini of peptides may influence their aggregation and secondary structure in water in both synthetic and natural systems. A hydrophobic, hydrocarbon and/or alkyl tail component with a sufficient number of carbon atoms coupled to an ionic peptide having a preference for beta-strand conformations can in certain embodiments be used to create an amphiphile that assembles (e.g., in vivo) into nanofiber structures. The amphiphile's overall conical shape can also have an effect on such assemblies. Self-assembling may be triggered by body fluid (e.g., cerebral spinal fluid).

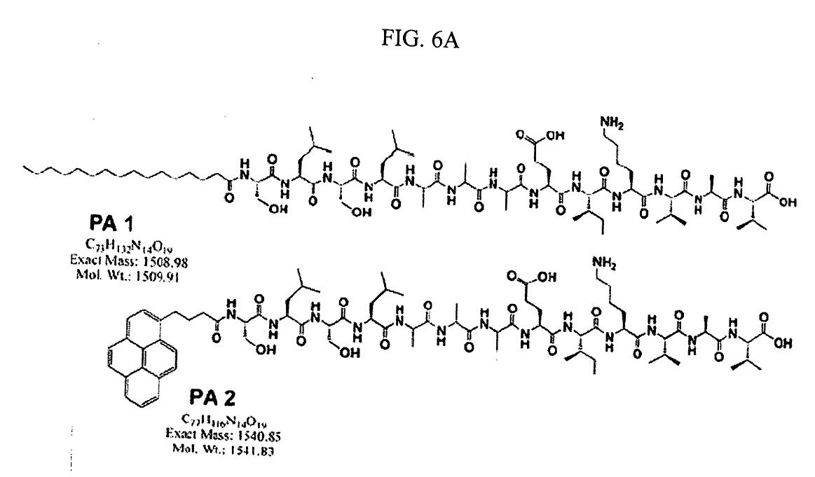

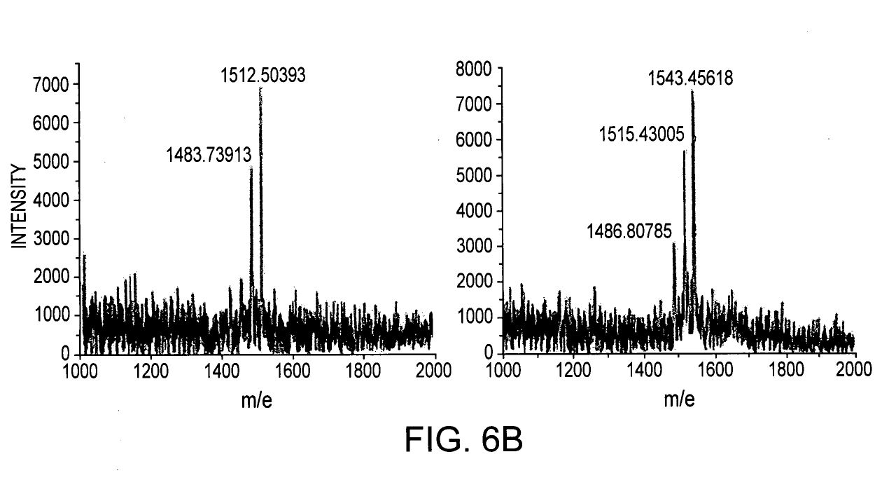

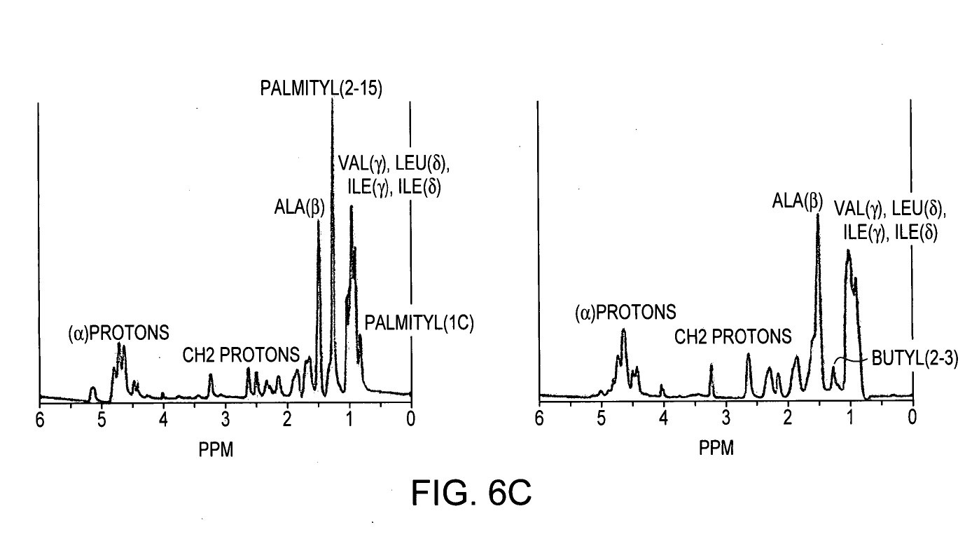

The present invention is not limited by the peptide amphiphile(s) utilized. Indeed, a variety of peptide amphiphiles are contemplated to be useful in the present invention including; but not limited to, those described in U.S. Pat. Apps. 20050272662, 20050209145, 20050208589, 20040258726, 20040022718, 20040018961, 20040001893, and international applications WO/05056576, WO/05056039, WO/05003292, WO/04106359, WO/04072104, WO/04046167, WO/04018628, WO/04003561, WO/03090255, WO/03084980, WO/03070749, WO/03054146 (each of which is hereby incorporated by reference in its entirety), as well as those described herein (e.g., in Examples 1 through 13, and FIGS. 6, and 14-17.

In some preferred embodiments, a peptide amphiphile (PA) of the present invention comprises an organic moiety (e.g., comprising a hydrophobic region (e.g., a linear peptide chain (e.g., a palmitoyl group) or a hydrophobic ring structure (e.g., pyrenebutyl))) joined to a structural region (e.g., comprising sequences (e.g., β-sheets) that can alter and/or influence packing and self-assembly of the peptide amphiphile) joined to a functional region (e.g., comprising a peptide epitope (e.g., IKVAV and/or YIGSR sequence). The peptide moiety may comprise one or more other regions (e.g., charged amino acid or sequence thereof (e.g., adjacent to the hydrophobic region, structural region or functional region)) that can determine the charge of the peptide amphiphile. Upon application of or exposure to a trigger (e.g., a change in pH or ion concentration (e.g., accomplished by exposure to cerebral spinal fluid, cell culture media, etc.) PA molecules self-assemble in an aqueous medium into nanofibers. In some embodiments, oppositely charged PA can be mixed in situ to create an electrostatically stabilized gel (e.g., comprising one or more than one type of peptide epitope).

In various preferred embodiments, the hydrophobic component of such a compound or composition is of sufficient length to provide amphiphilic behavior and nanofiber assembly/formation (e.g., in vivo or at physiologic pH). Typically, such a component may be about a C6 or greater hydrocarbon moiety, although other hydrophobic, hydrocarbon and/or alkyl components could be used as would be well-known to those skilled in the art to provide similar structural or functional effect. Such hydrophobic components include, without limitation, cholesterol, biphenyl and p-aminobenzoic acid.

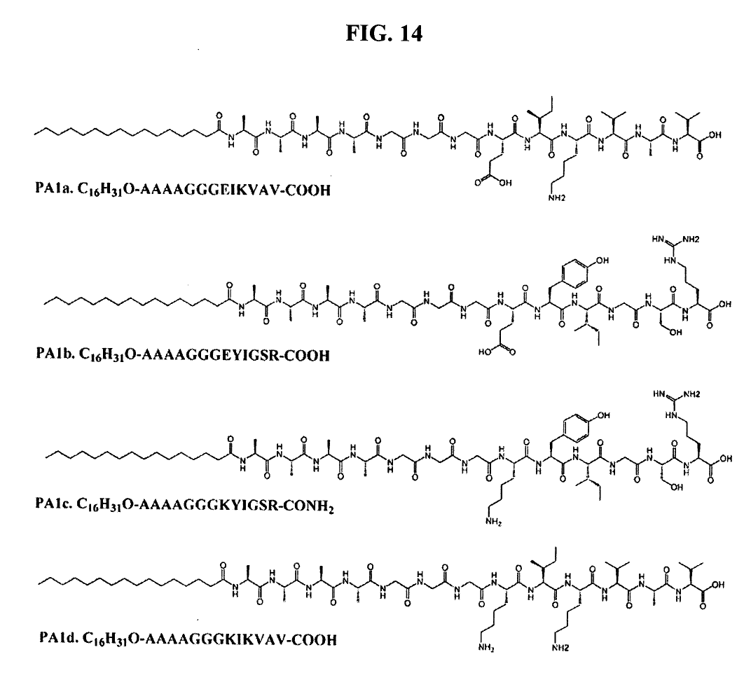

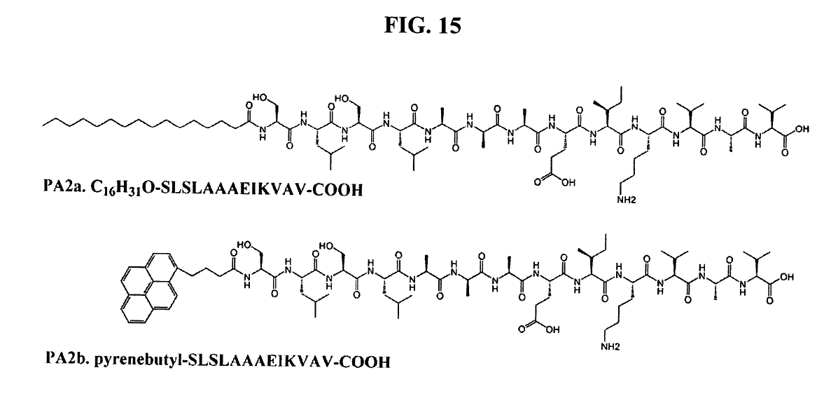

Some PAs form a strong, virtually instantaneous gel when it comes in contact with cerebrospinal fluid (e.g., the PA shown at the top of FIG. 14). During development of the present invention, attempts to inject a dilute solution of this molecule into the mouse spinal cord led to clogging of the small-bore needle used. Accordingly, this problem was overcome by making several modifications in an effort to promote slower self-assembly. First, the A4 section was replaced with an SLSL sequence. This alternating polar-nonpolar sequence was intended to lessen the hydrophobic driving force for self assembly and make favorable packing more difficult. The flexible G3 sequence was replaced with a stiffer A3, again to hinder packing (See, e.g., FIG. 15, top and bottom PAs). Gelation of these PAs was in fact slower (˜3-5 minutes) and less robust than that of the original PA molecule, as measured by visual observation and oscillatory rheometry. The PA on the bottom of FIG. 15 is identical to that depicted on the top of FIG. 15 except for the substitution of a pyrenebutyl tail for the palmityl tail. This change makes the PA molecules fluorescent and therefore suitable for tracking the PA in histological sections. Thus, in some embodiments, the PA may comprises a fluorescent region (e.g., a pyrenebutyl tail) for visual and tracking purposes.

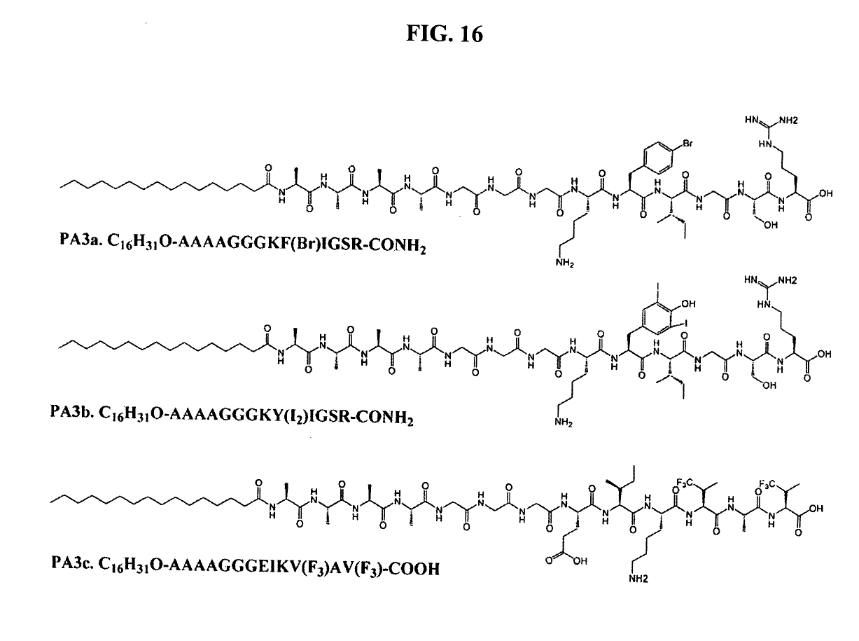

Additionally, a PA may be configured with the inclusion of a heteroatom (e.g., Br, I, or F) to provide a tag for distinguishing a particular PA from another PA with which it has coassembled, or from other peptides and proteins in the physiological environment. For example, FIG. 16 shows three such PAs. For example, the PA depicted at the top of FIG. 16 comprises a bromophenylalanine in place of tyrosine, replacing the hydroxyl group on carbon 4 with a bromine atom. The PA in the middle of FIG. 16 has an iodine added to the 3 and 5 positions on the ring. In some embodiments, bromine and iodine can be used due to their x-ray scattering properties. The PA depicted at the bottom of FIG. 16 comprises substitution of six valine gamma protons with fluorine atoms. In some embodiments, fluorine is used because its rarity in natural tissues and can be identified using EDX.

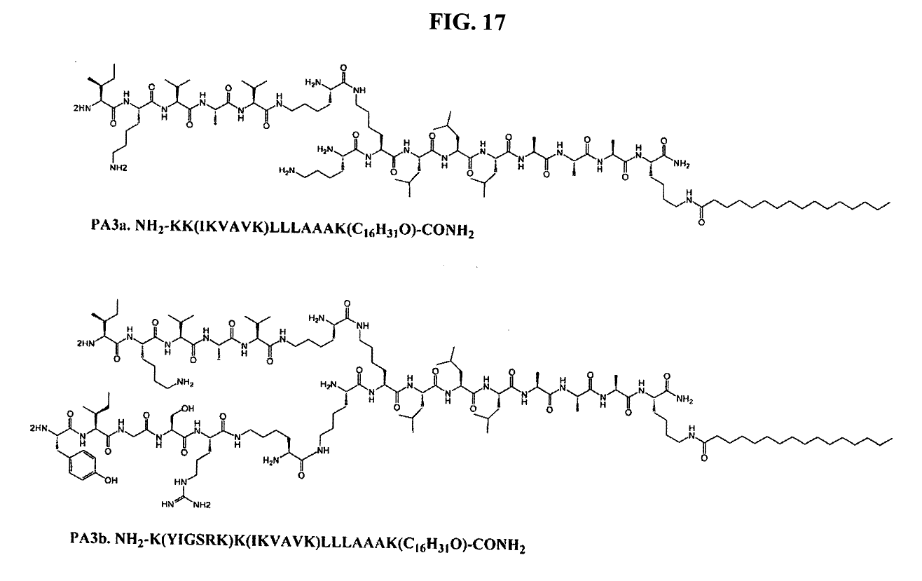

Alternatively, a PA may comprise one or more branching groups. In some embodiments, branching groups within a PA improves the availability and/or exposure of the peptide epitopes (e.g., to a target (e.g., a neuron)). In some embodiments, a PA with one or more branching groups has a modified lysine residue at its N-terminus (e.g., with a palmityl tail attached by a peptide bond to the epsilon carbon). In some embodiments. the N-terminus is chosen to be an amide rather than a free amine in order to maintain more hydrophobicity in the region. In some embodiments, a beta-sheet-promoting A3L3 sequence is attached to the C-terminus of the lysine, followed by a second modified lysine to which a peptide epitope (e.g., IKVAV or YIGSR) sequence is appended. Thus, in this embodiment, the I rather than the V is furthest from the tail in order to maintain proper chirality in the reversed synthesis scheme. The PA depicted on the top of FIG. 17 is exemplary of such a PA and has a free lysine added to the main backbone of the molecule at the N-terminus; whereas, the PA depicted on the bottom of FIG. 17 shows a YIGSR sequence appended to this lysine. In some embodiments, a PA formulated in this way is strongly positively charged and soluble only at low pH; thus, when the pH is adjusted to the physiological range they form gels.

In some embodiments, a composition comprising a PA may also comprise or be administered with one or more growth factors (e.g., neurotrophic factors (e.g., such that, when administered to a subject (e.g., via injection of a solution comprising the PA), the PA forms a nanofiber gel comprising the neurotrophic factor). Neurotrophic factors are a broad set of peptide growth factors that regulate development and survival of neurons of the central nervous system (CNS) and the peripheral nervous system (See, e.g., Huang and Reichardt. 2001 Annu. Rev. Neurosci. 24:677-736; Neet et al., 2001 Cell. Mol. Life Sci. 58:1021-1035). The present invention is not limited by the type of growth factor used. A variety of neurotrophic factors can be used including, but not limited to, Nerve growth factor (NGF), Brain-derived neurotrophic factor (BDNF), Neurotrophin-3 (NT-3), Neurotrophin-4/5 (NT-4/5), Ciliary neurotrophic factor (CNTF), Leukemia inhib. factor=chol. neuronal diff. factor (LIF/CDF), Cardiotrophin-1, Basic fibroblast growth factor (bFGF), Acidic fibroblast growth factor (aFGF), Fibroblast growth factor-5 (FGF-5), Insulin, Insulin-like growth factor I (IGF-I), Insulin-like growth factor Ii (IGF-II), Transforming growth factor β1 (TGFβ1), Transforming growth factor β2 (TGFβ2), Transforming growth factor β3 (TGFβ3), Activin, Glial cell-derived neurotrophic factor (GDNF), MidkineHeparin-binding neurotrophic factor (HBNF), Pleiotrophin, Epidermal growth factor (EGF), Transforming growth factor α (TGFα), Schwannoma-derived growth factor, Heregulin (neuregulin, ARIA), Interleukin 1, Interleukin 2, Interleukin 3, Interleukin 6, Axon ligand-1 (Al-1), elf-1, ehk1-L, and LERK2, as well as factors being evaluated in clinical trials.

In some embodiments, a composition comprising a PA may also comprise or be administered with one or more agents that inhibit (e.g., blocks) activity and/or expression of a neuronal growth inhibitor (e.g., such that, when administered to a subject (e.g., via injection of a solution comprising the PA), the PA forms a nanofiber gel comprising the inhibitor of a neuronal growth inhibitor). The present invention is not limited by the type of inhibitor utilized. Indeed, a variety of inhibitors can be used including, but not limited to, myelin inhibitors, Nogo, Ryk and Ryk-like inhibitors, sFRP and sFRP-like substances, MAG, Omgp, and Wnt inhibitors. Other inhibitors present in glial scar, such as CSPG, also inhibit axonal outgrowth. It is not fully understood whether CSPG are the actual active components for the inhibitors of axonal regeneration or other molecules associate with CSPG are the active components. Indeed, the present invention contemplates inhibiting any inhibitor that prevents axonal growth after injury. Those of skill in the art will understand that there are many manners in which such inhibitors can be blocked, and will, by following the teachings contained herein, be able to develop means to block these inhibitors in the context of the invention. For example, inhibitors of axonal growth inhibitors may comprise antibodies and/or siRNAs specific for the inhibitors (e.g., expressed from an expression vector or cassette included in a composition comprising a PA of the present invention).

In some embodiments, a composition comprising a PA may also comprise or be administered with one or more agents that attract neuronal growth including, but not limited to, a Wnt, Netrin, Shh, cell adhesion molecule, Ig superfamily member, cadherin, integrin, EphrinB, ECM molecule or HGF. In some embodiments, a composition comprising a PA may also comprise or be administered with one or more agents that repel neuronal growth including, but not limited to, a Semiphorin, Netrin, Slit, Wnt, BMP, Ephrin or member of the Ig superfamily.

Additionally, there are many protein attractants and repellants that play a role in axonal guidance. Further, many such axon guidance molecules are bi-functional: attractive to one type of axons and repulsive to another, depending on the receptor composition in the responding growth cones.

A number of molecules direct axonal growth during development. These compounds are play important roles in embryonic development, and may function in the same or a similar way in the adult CNS.

Attractants and repellants can be divided into two general categories, diffusable and non-diffusable. Diffusible attractants include, but are not limited to, Netrins, Shh, Wnts, and HGF. Diffusible repellents include, but are not limited to, secreted Semaphorins, Netrins, Slits, Wnts, and BMPs. Non-diffusible attractants include, but are not limited to: cell adhesion molecules such as members of the Ig superfamily, Cadherins, and Integrins; Ephrins; and ECM molecules. Non-diffusable repellents include, but are not limited to, Ephrins, members of the Ig superfamily, and membrane-bound Semaphorins.

Those of skill in the art will be able to use these, and any other attractants or repellants in the context of the invention. For example, those of skill in the art will be able to generate a composition comprising a PA comprising one or more of these agents. Furthermore, such a composition could be administered to a subject in order to promote neurite growth in the subject (e.g., at a site of injury (e.g., spinal cord injury) or disease (neuronal degradation caused by the disease (e.g., diabetes)).

In the context of the invention, native attractants or repellants may be employed. Further, proteins, polypeptides, peptides, mutants, and/or mimetics of these attractants or repellants may be employed.

In some embodiments, a composition comprising a PA may comprise one or more peptide epitopes. The present invention is not limited by the type of peptide epitope utilized. In some embodiments, the epitope is any neurobioactive epitope present within laminin (herein referred to as a “laminin epitope”, e.g., that stimulates development of neurons). In some preferred embodiments, the peptide epitope is a IKVAV sequence. IKVAV is a laminin sequence known to interact with mammalian neurons. IKVAV promotes neurite outgrowth in mammalian neurons. The present invention is not limited to the use of IKVAV. Other suitable bioactive epitopes find use in the methods of the present invention (e.g., a YGSIR sequence). The peptide components (e.g., peptide epitopes) of the invention preferably comprise naturally-occurring amino acids. However, incorporation of known artificial amino acids such as beta or gamma amino acids and those containing non-natural side chains, and/or other similar monomers such as hydroxyacids are also contemplated, with the effect that the corresponding component is peptide-like in this respect.

In some embodiments, a mimetic of a laminin epitope is utilized. As used herein, a “mimetic of a laminin epitope” is intended to refer to any molecule other than a native sequence of laminin (e.g., IKVAV) that is able to maintain an acceptable level of equivalent biological activity as a native laminin epitope.

It is well understood by the skilled artisan that, inherent in the definition of a “mimetic of a laminin epitope,” is the concept that there is a limit to the number of changes that may be made within a defined portion of the molecule and still result in a molecule with an acceptable level of equivalent biological activity (e.g., the ability of IKVAV sequences to modulate neuronal growth and regeneration). “Mimetic of a laminin epitope” is thus defined herein as any laminin epitope polypeptide in which some, or most, of the amino acids may be substituted so long as the polypeptide retains substantially similar activity in the context of the uses set forth herein. Of course, a plurality of distinct proteins/polypeptides/peptides with different substitutions may easily be made and used in accordance with the invention. Additionally, in the context of the invention, a mimetic of a laminin epitope can be a laminin epitope homologue polypeptide from any species or organism, including, but not limited to, a human polypeptide. One of ordinary skill in the art will understand that many mimetics of a laminin epitope would likely exist and can be identified using commonly available techniques.

Amino acid sequence mutants of a laminin epitope also are encompassed by the present invention. Amino acid sequence mutants of a laminin epitope of any species, such as human and mouse laminin epitope, is contemplated by the present invention. Amino acid sequence mutants of a laminin epitope can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide.

Amino acid substitutions are generally based on the relative similarity of the amino acid side-chain substituents (e.g., their hydrophobicity, hydrophilicity, charge, size, and the like). An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape.

In making changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (See, e.g., Kyte and Doolittle, 1982, incorporated by reference herein in its entirety). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those which are within +1 are particularly preferred, and those within +0.5 are even more particularly preferred.

It is understood that an amino acid can be substituted for another having a similar hydrophilicity value and still obtain a biologically equivalent protein. As detailed in U.S. Pat. No. 4,554,101, herein incorporated by reference in its entirety, the following hydrophilicity values have been assigned to amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +2 is preferred, those which are within +1 are particularly preferred, and those within +0.5 are even more particularly preferred.

In some embodiments, a composition comprising a PA may also comprise or be administered with one or more neuroprotective agents (e.g., a buckyball-type agent shown to lessen the aftereffects of stroke, head trauma and spinal cord injury).

In some embodiments, the PA compositions form a sol-gel system including 1) a polar or aqueous solution and/or containing of one or more of the amphiphile compounds or compositions described herein, and 2) a factor or reagent sufficient to induce assembly, agglomeration of gelation under neutral or physiological conditions. Such gelation and/or self-assembly of various PA compositions into micellular nanofibers can be achieved under substantially neutral and/or physiological pH conditions through drying, introduction of a mono- or multivalent metal ion and/or the combination of differently charged amphiphiles.

II. Methods of Using Compositions Comprising a Peptide Amphiphile (PA) of the Present Iinvention

Experiments conducted during the course of development of the present invention demonstrated that neural progenitors cells were able to be efficiently differentiated into neurons using the methods of the present invention (See, e.g., Example 1-8). The cells demonstrated differentiation without formation of significant amounts of astrocytes (See, e.g., Examples 6 and 11). Furthermore, experiments demonstrated that administration (e.g., injection into an injured spinal cord) of a composition comprising a PA of the present invention to a subject with an injured spinal cord reduces astrogliosis at the site of injury, promotes substantial regeneration of sensory and motor fibers, and significantly enhances behavioral recovery (e.g., mobility of limbs paralyzed prior to such treatment (See, e.g., Examples 1, 9-13).

In some embodiments, the methods and compositions of the present invention find use in encapsulation of a variety of cell types. The present invention is not limited to a particular cell type. Examples include, but are not limited to primary cell cultures, stem cells (e.g., human or non human) and other pluripotent cell lines, progenitor cells, neurites and other neurons at different stages of development, and immortalized cell lines. The peptide-amphiphile compositions of the present invention are also suitable for use with tissue and animals. Cells treated with (e.g., administered) compositions of the present invention may be damaged cells (e.g., damaged neurons) or generally healthy cells (e.g., neurons (e.g., neurons that are treated in order to generate neurons with greater than normal axonal signaling (e.g., motor or sensory) capabilities).

In some embodiments, the methods of the present invention are utilized in the encapsulation of stem cells. The methods of the present invention are suitable for use with a variety of stem cells including, but not limited to, embryonic stem cells and adult stem cells. Embryonic stem cells may be obtained from a variety of sources including, but not limited to, embryonic stem cell lines and embryonic germ cell lines derived from primordial germ cells (PGCS) cells isolated, according to one embodiment, from gonadal tissues, genital ridges, mesenteries or embryonic yolk sacs of human embryos (See, e.g., U.S. Pat. No. 6,562,619). Embryonic stem cells may also be obtained from commercial or research sources, including, but not limited to, those described above. Adult stem cells may be derived from a variety of cell types, including, but not limited to, those disclosed herein.

The encapsulated cells of the present invention find use in the proliferation and differentiation of the cells (e.g., neurites). As described above, the nature of nanofiber gels (e.g., self-assembled using a composition comprising a PA of the present invention) allows for the delivery of bioactive reagents (e.g., that induce differentiation or proliferation) at high local (e.g., near van der Waals) concentrations. Any bioactive agent (e.g., peptide) for which it is desirable to form high local concentrations can be delivered using the methods and compositions of the present invention. For example, in some embodiments, hormones (e.g., peptides), growth factors, differentiation factors, or other proteins or small molecules are incorporated into a composition comprising a PA of the present invention (e.g., to generate a nanofiber gel comprising the bioactive agent). One skilled in the relevant arts recognizes that other agents (e.g., peptides) may be utilized with the methods and compositions of the present invention.

The present invention is not limited by the condition (e.g., injury or disease) treated with the compositions and methods of the present invention. In some preferred embodiments, compositions and methods of the present invention are used to treat nerve damage (e.g., caused by traumatic injury (e.g., spinal cord injury)). In some preferred embodiments, treatment comprises administering a composition comprising a PA (e.g., comprising IKVAV) under conditions such that axonal growth (e.g., regeneration) occurs.

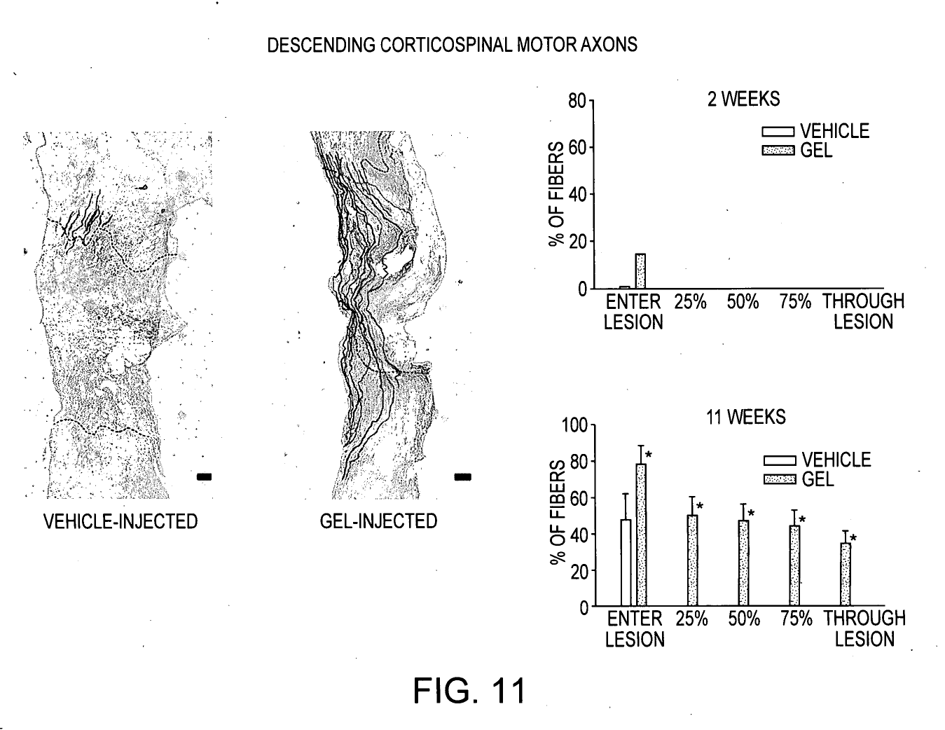

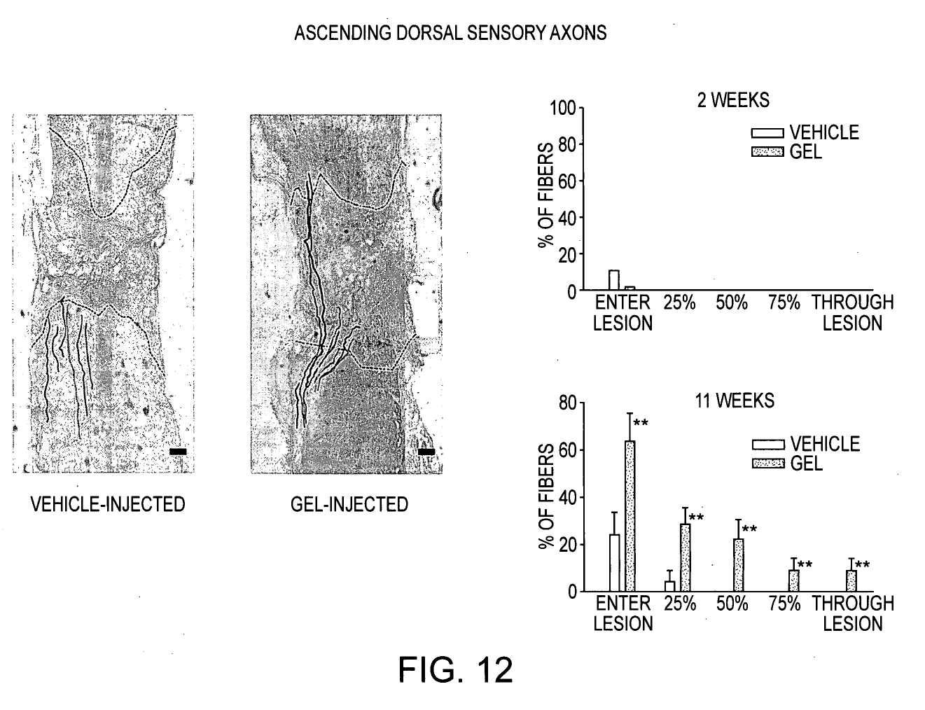

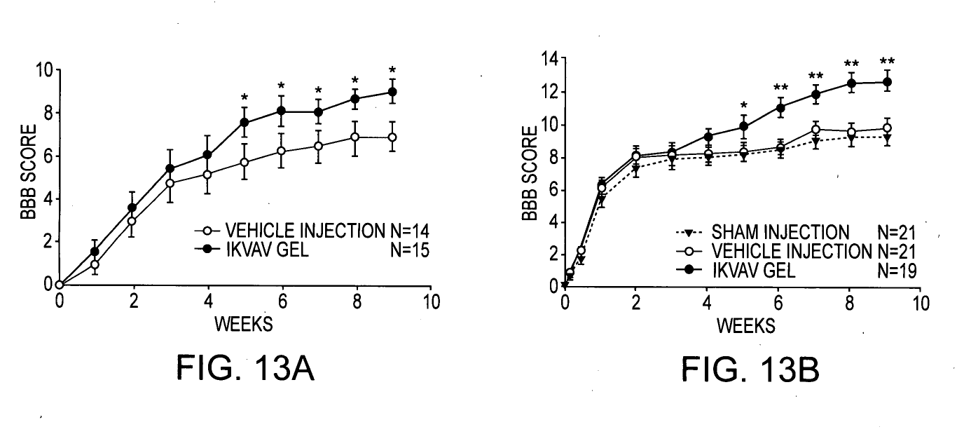

In some embodiments, the subject may have a disorder of the spinal cord. Any disorder of the spinal cord is contemplated by the present invention. In certain embodiments, the disorder of the spinal cord is traumatic spinal cord injury (discussed above). For example, in some preferred embodiments, compositions and methods of the present invention promote regeneration of motor axons and of sensory axons following spinal cord injury (See, e.g., Examples 12 and 13, FIGS. 11 and 12). In some preferred embodiments, regeneration of motor axons and sensory axons in a subject with a spinal cord injury leads to anatomic improvements in the treated subject (e.g., movement of a limb paralyzed (e.g., partially or fully) prior to treatment (See, e.g., Example 13 and FIG. 13). The traumatic spinal cord injury may or may not have resulted in paralysis of the subject. The neuronal dysfunction can be by any mechanism. For example, cell death can be the result of acute traumatic injury or degeneration.

Any disease or condition wherein there is neuronal dysfunction is contemplated by the present invention. In addition to spinal cord injury, other examples include Parkinson's disease, where dopaminergic neurons undergo degeneration and ALS where neurons in the motor systems undergo degeneration. In these cases, stem cells are being developed so that they can be transplanted to the midbrain and the spinal cord, respectively, so that they can populate and make proper connection with their targets. The establishment of new connections require the growth of axons from these neural stem cells. Compositions and methods of the present invention can be used in growth and guidance of regenerating axons from these stem cells.

In some embodiments, nerve damage treated with compositions and methods of the present invention is associated with a lesion or a disease or dysfunction of the nervous system. In some embodiments, the nerve damage results from a spinal cord injury, head trauma or stroke. In some embodiments, the nerve damage results from a neurodegenerative disease. In some embodiments, the nerve damage results from chemical injury or as a result of chemotherapy. In some embodiments, the nerve damage is diabetic neuropathy.

Diabetic neuropathies are a family of nerve disorders caused by diabetes. People with diabetes can, over time, have damage to nerves throughout the body. Neuropathies lead to numbness and sometimes pain and weakness in the hands, arms, feet, and legs. Problems may also occur in every organ system, including the digestive tract, heart, and sex organs. People with diabetes can develop nerve problems at any time, but the longer a person has diabetes, the greater the risk. Diabetic neuropathies can be classified as peripheral, autonomic, proximal, and focal. Each affects different parts of the body in different ways.

Peripheral neuropathy causes either pain or loss of feeling in the toes, feet, legs, hands, and arms. Autonomic neuropathy causes changes in digestion, bowel and bladder function, sexual response, and perspiration. It can also affect the nerves that serve the heart and control blood pressure. Autonomic neuropathy can also cause hypoglycemia (low blood sugar) unawareness, a condition in which people no longer experience the warning signs of hypoglycemia. Proximal neuropathy causes pain in the thighs, hips, or buttocks and leads to weakness in the legs. Focal neuropathy results in the sudden weakness of one nerve, or a group of nerves, causing muscle weakness or pain. Any nerve in the body may be affected. Thus, in some embodiments, the present invention provides compositions (e.g., comprising a PA) and methods of treating diabetic neuropathy (e.g., regenerate nerve function to nerves damaged as a result of diabetes) and/or treat signs and symptoms of diabetic neuropathy (e.g., digestive problems such as feeling full, nausea, vomiting, diarrhea, or constipation, problems with bladder function, problems having sex, dizziness or faintness, loss of the warning signs of low blood glucose, increased or decreased sweating or changes in how eyes react to light and dark).

In some embodiments, compositions and methods of the present invention are utilized in conjunction with cell transplantation or other strategies (See, e.g., Wald et al., Biomaterials 14, 270 (1993); Yannas, Science 215, 174(1982); Mooney et al., Biomaterials 17, 1417 (1996); Mikos et al., Biomaterials 15, 55 (1994); Lavik et al., Methods Mol. Biol. 198, 89 (2002); Hsu et al., Invest. Ophthalmol. Vis. Sci. 41, 2404 (2000); Chamberlain et al., J. Neurosci. Res. 60, 666 (2000); Butler et al., Br. J. Plast. Surg. 52, 127 (1999); Powell et al., J. Neurosci. Res. 61, 302 (2000); Cornish et al., Mol. Cell. Neurosci. 20, 140 (2002)) thereby enhancing its therapeutic efficacy.

In some embodiments, compositions and methods of the present invention are used to treat (e.g., regenerate nerve function to) lingual nerve damage. Lingual nerve injury or damage can result in anesthesia (numb tongue), paresthesia (tingling), or dysesthesia ( pain and burning ) in the tongue and inner mucosa of the mouth. This can be due to complication of tooth extraction of the wisdom teeth ( third molar) or dental anesthetic injection (nerve block) for fillings, crowns. It results in a chronic pain syndrome or neuropathy. If the inferior alveolar nerve is involved, numbness of the lip may result.

In some embodiments, compositions and methods of the present invention are used to treat injury to (e.g., regenerate nerve function to) the inferior alveolar nerve. Injury to the inferior alveolar nerve can result in anesthesia, paresthesia, or dysesthesia of the chin, lower lip, and the jaw. This nerve can be injured by injection, but is more commonly injured during wisdom tooth extraction. It can also be injured by root canal procedures, other tooth extractions and with placement of implants.

In some embodiments, compositions and methods of the present invention are utilized to treat nerve damage (e.g., regenerate nerve function to) that is a complication of peripheral nerve block. In some embodiments, compositions and methods of the present invention are utilized to treat damage (e.g., regenerate nerve function to) of any one or more cranial nerves.

In some embodiments, the compositions and methods of the present invention are utilized to treat auditory neuropathies (e.g., regenerate nerve function to nerves damaged as a result of auditory neuropathy). Several types of nerve damage accompany auditory neuropathy including focal primary demyelination, diffuse primary demyelination, axonal loss, and axonal loss with secondary demyelination and remyelination. Changes in auditory nerve discharges with neuropathies include a disorder of temporal synchrony for diffuse primary demyelination and axonal loss with secondary demyelination and remyelination. Changes in temporal encoding account for a subjects' impairment on auditory tasks requiring precise encoding of temporal cue such as speech comprehension, localization of sound sources, and gap detection. Neuropathies are also associated with a change in nerve fiber excitability limiting the rate of discharge. Both axonal and demyelinating diseases are accompanied by impaired excitability of affected fibers with the extreme being conduction block in demyelinating disorders and hyperpolarization block in axonal disease.

In some embodiments, compositions and methods of the present invention are utilized in the treatment or prevention of disorders or diseases of the CNS, brain, and/or spinal cord. These disorders can be neurologic or psychiatric disorders. These disorders or diseases include brain diseases such as Alzheimer's disease, Parkinson's disease, Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. Compositions and methods of the present invention can also be utilized to treat subjects suffering from or at risk for nerve damage from cerebrovascular disorders such as stroke in the brain or spinal cord, from CNS infections including meningitis and HIV, from tumors of the brain and spinal cord, or from a prion disease. Compositions and methods of the present invention can also be employed to deliver agents to counter CNS disorders resulting from ordinary aging (e.g., anosmia or loss of the general chemical sense), or brain injury of any kind.

Compositions and methods of the present invention can also be utilized to treat nerve tissue damage following radical pelvic surgeries (e.g., prostatectomy, particularly, post-radical prostatectomy, in which the nerve tissue (e.g., cavernous nerve tissue and/or pelvic nerve tissue) becomes damaged).

Thus, in some embodiments, compositions and methods of the present invention promote neuronal survival and regeneration and can also can support the innervation of tissue that is, for example, damaged, injured, diseased, or transplanted, thus allowing repair of the nerve tissue, along with providing treatment and improvement of an associated dysfunction (e.g., erectile dysfunction, bladder voiding) following surgery.

The present invention contemplates using compositions and methods described herein for both the therapy and prophylaxis of diseases or injuries where nerve damage occurs. In a therapeutic context, such situations include, but are not limited to diseases including peripheral nerve damage, such as by physical injury or disease state such as diabetes, in the case of injury or a disease state of the CNS, including physical damage to the spinal cord, brain trauma, stroke, retinal and optic nerve lesions, neurodegenerative diseases such as Alzheimer's disease and Parkinson's disease, neuromuscular diseases, autoimmune diseases of the nervous system, tumours of the central nervous system, damage to motor neurons such as occurs in conditions such as amyotrophic lateral sclerosis, and degenerative diseases of the retina such as retinitis pigmentosa and age-related macular degeneration.

As will be appreciated by one of skill in the art, the present invention is not limited to any particular site of nerve damage that is treated (e.g., via regeneration of nerve function (e.g., through stimulation of axonal growth)) using the compositions and methods of the present invention. Indeed, a nerve to be treated, and corresponding regaining of function of a body part innervated by the nerve, may be one found, for example, in the spine, hand, leg, arm, back, finger, face, head, neck, tongue, ear, penis, foot, toe, eye, or mouth of a subject.

Compositions and methods of the present invention can be used to alter (e.g. modulate) the growth of a neuron (e.g., at any stage in development (e.g. a neurite or a mature neuron)). The methods for modulating growth of a neuron may, in certain embodiments, be methods for stimulating growth of a neuron, methods for regenerating a damaged neuron, or methods for guiding growth of a neuron.

The neuron to be modulated may be any neuron. In some embodiments, the neuron is a neuron in the spinal cord that has been damaged. For example, the spinal cord may have been damaged by traumatic spinal cord injury. The damage may have resulted in impaired function of the neuron.

In some embodiments, the method for modulating growth of a neuron is a method for modulating growth of a neuron in a subject. Although any subject is contemplated by the present invention, in certain embodiments the subject may be a subject with a disorder of the spinal cord. The disorder of the spinal cord may be any disorder, such as a traumatic spinal cord injury. The traumatic spinal cord injury may or may not have resulted in paralysis of the subject. In further embodiments, the patient is a patient with a neurodegenerative disease. The neuron to be modulated can be a sensory or a motor neuron.

In some embodiments, compositions and methods of the present invention can be utilized in research applications (e.g., to understand the effect of peptides on cell (e.g., neural progenitor, neurites or other neuronal cell) differentiation and proliferation). In other embodiments, compositions and methods of the present invention can be utilized in drug screening (e.g., to screen candidate peptides).

For example, the present invention contemplates the screening of candidate substances for the ability to modulate growth of a cell (e.g., a neuron, neurite or other type of neuronal cell). Particularly preferred candidate substances will be those useful in stimulating axonal growth within the spinal cord. In the screening assays of the present invention, the candidate substance may first be screened for basic biochemical activity and then tested (e.g., when placed within a composition comprising a PA of the present invention (e.g., a nanofiber gel self-assembled from a composition comprising a PA and a candidate substance) for its ability to modulate activity, at the cellular, tissue or whole animal level. In certain embodiments, an explant assay such as an assay using cultured spinal cord sections may be used in the screening methods. Any method known to those of skill in the art may be used in the claimed invention to conduct the screening assays.

In some embodiments, the present invention provides methods of screening for modulators of growth of a neuron. In some embodiments, the present invention is directed to a method of obtaining a candidate substance; combining or co-administrating the candidate substance with a PA of the present invention; contacting the candidate substance (e.g., combined with or co-administered with a PA of the present invention) with a neuron; and measuring modulation of growth of the neuron (e.g., axonal (e.g., motor or sensory) growth). In some embodiments, a candidate substance can be identified as an inhibitor or an activator of neuron growth. An inhibitor according to the present invention may be one which exerts an inhibitory effect on the growth of a neuron (e.g., as measured by the methods disclosed herein). An activator according to the present invention may be one which exerts a stimulatory effect on the growth of a neuron.

As used herein, the term “candidate substance” refers to any molecule that may potentially modulate (e.g., stimulate or inhibit) regeneration of a neuron. The candidate substance may be a protein or fragment thereof, a polypeptide, a peptide, a small molecule inhibitor, or even a nucleic acid molecule (e.g., expressed by an expression vector). It may prove to be the case that the most useful pharmacological compounds will be compounds that are structurally related to compounds that interact with the activators and inhibitors of neuron (e.g., axonal growth) described herein, or chemical compounds affecting signaling pathways related to the activators and inhibitors. Creating and examining the action of such molecules is known as “rational drug design,” and includes making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a known activator or inhibitor (e.g., those described herein) and then design a molecule for its ability to interact with the activator or inhibitor. Alternatively, one could design a partially functional fragment of an activator or inhibitor or a like substance (binding, but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore.

On the other hand, one may simply acquire, from various commercial sources, small molecule libraries that are believed to meet the basic criteria for useful drugs in an effort to identify useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known modulators of neuronal growth.

Other suitable inhibitors include antisense molecules (e.g., siRNAs (e.g., expressed from an expression vector), ribozymes, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

In some embodiments, the present invention provides a pharmaceutical composition comprising a peptide amphiphile, wherein the peptide amphiphile is configured to alter (e.g., stimulate) neuron (e.g., neurite) growth. Any type of pharmaceutical preparation of a peptide amphiphile of the present invention (e.g., a composition comprising a peptide amphiphile, or a composition comprising a peptide amphiphile and one or more other agents (e.g., a known stimulator or inhibitor of neuron growth, a growth factor, a neurotrophic factor, a compound identified by the methods of the present invention as being an activator or inhibitor, etc.) is contemplated by the current invention. One of skill in art would be familiar with the wide range of types of pharmaceutical preparations that are available, and would be familiar with skills needed to generate these pharmaceutical preparations.

In some embodiments, the pharmaceutical preparation will be an aqueous composition (e.g., those described in Examples 1, 3 and 11 (e.g., diluted in a glucose solution)). Aqueous compositions of the present invention comprise an effective amount an of a peptide amphiphile dissolved or dispersed in a pharmaceutically acceptable carrier (e.g., glucose or saline solution) or aqueous medium.

As used herein, “pharmaceutical preparation” includes any and all solvents, dispersion media, coatings, antibacterial and antifungal agents,. isotonic and absorption delaying agents and the like. The use of such media and agents for pharmaceutical active substances is well known in the art. Except insofar as any conventional media or agent is incompatible with the active ingredient (e.g., the peptide amphiphile), its use in a therapeutic compositions is contemplated. Supplementary active ingredients can also be incorporated into the compositions (e.g., those describe herein (e.g., growth factors, neurotrophic factors, and inhibitors of inhibitors of neuron growth). For human administration, preparations should meet sterility, pyrogenicity, general safety and purity standards as required by FDA Office of Biologics standards.

The biological material may generally be formulated for administration by any known route, such as through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like. The preparation of an aqueous composition containing an active agent (e.g., peptide amphiphile) of the invention disclosed herein as a component or active ingredient will be known to those of skill in the art in light of the present disclosure.

An agent or substance of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. A person of ordinary skill in the art would be familiar with techniques for generation of salt forms. The carrier can also be a solvent or dispersion medium containing, for example, water, ethanol, polyol (for example, glycerol, propylene glycol, and liquid polyethylene glycol, and the like), suitable mixtures thereof, and vegetable oils.

The present invention contemplates one or more peptide amphiphiles that will be in pharmaceutical preparations that are sterile solutions for parenteral injection or for application by any other route. A person of ordinary skill in the art would be familiar with techniques for generating sterile solutions for injection or application. Sterile injectable solutions are prepared by incorporating the active compounds in the required amount in an appropriate solvent with various of the other ingredients familiar to a person of skill in the art and by those disclose herein.

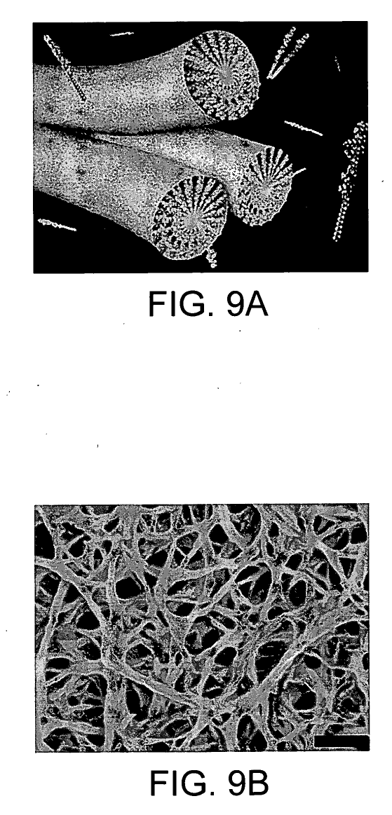

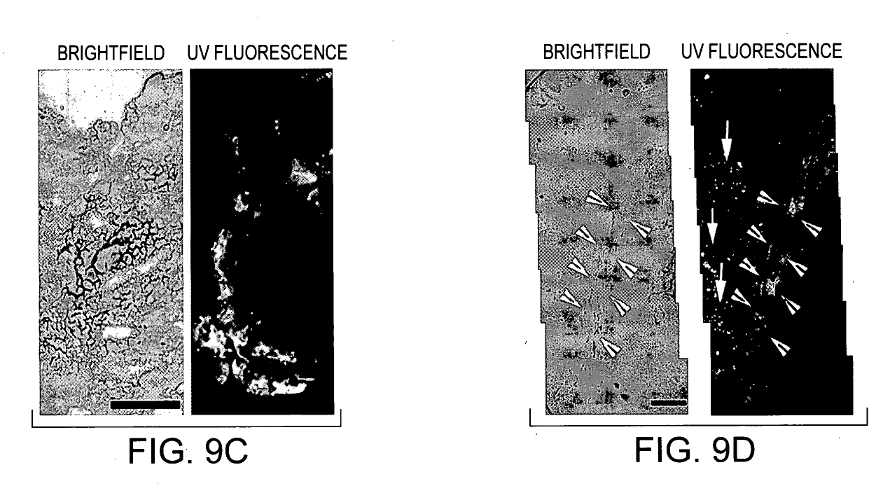

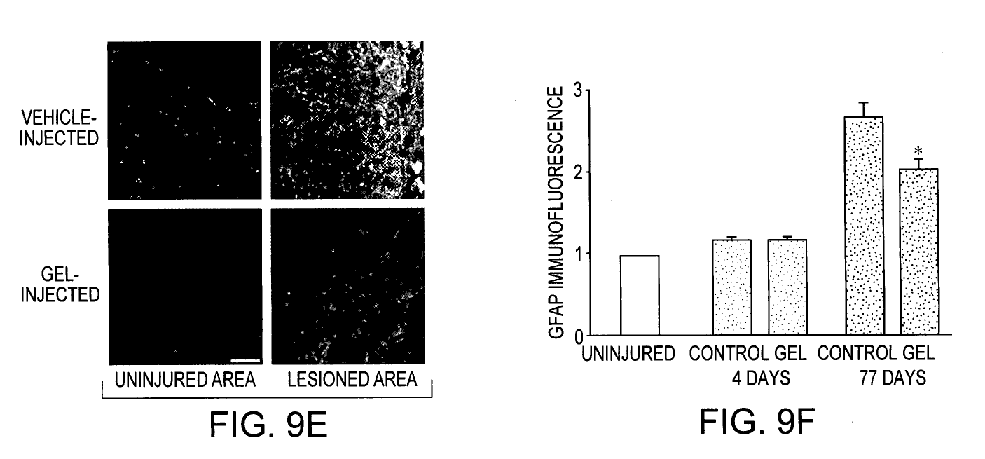

Upon formulation, solutions will be administered in a manner compatible with the dosage formulation and in such amount as is therapeutically effective. The formulations are easily administered in a variety of dosage forms, such as the type of injectable solutions described above. In some embodiments, the present invention provides a composition comprising a peptide amphiphile solution that, when placed in contact with a neuron (e.g., when injected into an injured spinal cord) forms a nanofiber gel, the nanofiber gel characterized in that it can exist for a period of time (e.g., for two of more days, for a week, for between one and two weeks, for more than two weeks, for between two and four weeks, or for more than four weeks (e.g., see Example 10 and FIG. 9)) within a subject.

In some embodiments, for parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Formulations for administration via lumbar puncture into the cerebrospinal fluid are also contemplated by the present invention.

The active agents disclosed herein may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. Doses capable of generating neuronal growth are described herein (e.g., See Examples 1, 3, 8 and 10).

An effective amount of the therapeutic or preventive agent is determined based on the intended goal, for example, axonal growth. The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For example, following traumatic spinal cord injury, a continuous administration of the therapeutic agent may be administered for a defined period of time, such as direct injection into the site of injury or into the cerebrospinal fluid near the site of injury. Continuous perfusion of the region of interest may be preferred. In other embodiments, compositions comprising PA of the present invention are configured such that they need only be administered once, twice, three, four or more times over a period of one, two, three, four or more (e.g., 8-52) weeks.

In order to increase the effectiveness of the compositions and methods disclosed herein, it may be desirable to combine a variety of agents into one or more pharmaceutical compositions that can be administered in a regime that is effective in the treatment of the neuronal injuries or disorders described herein. As discussed elsewhere in this specification, those of skill in the art may wish to apply a combination of neuronal attractive, repellant, inhibitory, and/or inhibition blocking substances to the neurons to facilitate appropriate neuronal growth and/or function. This may involve contacting the neuron or spinal cord with these agent(s) at the same time. This may be achieved by contacting the neuron or spinal cord with a single composition or pharmacological formulation that includes multiple agents (e.g., includes a composition comprising one or more peptide amphiphiles, or one peptide amphiphile and one or more other agents), or by contacting the cell with two distinct compositions or formulations, at the same time (e.g., a composition comprising a peptide amphiphile of the present invention co-administered with one or more separate compositions).

The agents may be applied to a neuron or spinal cord in series or succession at intervals ranging from minutes to weeks. In embodiments where two agents are applied separately to the neuron or spinal cord, one may wish ensure that a significant period of time did not expire between the time of each delivery, such that the agents will be able to exert an advantageously combined effect on the neuron(s). In such instances, it is contemplated that one may contact the cell with both modalities within about 12-24 hours of each other and, more preferably, within about 6-12 hours of each other. In some situations, it may be desirable to extend the time period for treatment significantly, however, where several days (2, 3, 4, 5, 6, 7 or more ) to several weeks (1, 2, 3, 4, 5, 6, 7, 8 or more) lapse between the respective administrations. In other embodiments, two or more agents are applied separately to a neuron or spinal cord in such a way that the agents are able to separately exert their beneficial therapeutic effects on the neurons. In such instances, it is contemplated that one may contact the cell with both modalities.

Various combinations, in an exemplary embodiment, may be employed. For example, any number of regimes may be employed as set forth below where “A” is a peptide amphiphile of the present invention and “B” a different peptide amphiphile, a growth factor, neurotrophic factor, a compound providing attractive or repellant guidance to neuronal growth, inhibitor of neuronal growth, blocker of an inhibitor of neuronal growth or other agent described herein:

A/B/A, B/A/B, B/B/A, A/A/B, A/B/B, B/A/A, A/B/B/B, B/A/B/B,
B/B/B/A, B/B/A/B, A/A/B/B, A/B/A/B, A/B/B/A, B/B/A/A, B/A/B/A,
B/A/A/B, A/A/A/B, B/A/A/A, A/B/A/A, and A/A/B/A.

Administration of the agents to a patient will follow general protocols for the administration as known to those of skill in the art and set-forth herein. It is expected that the treatment cycles may be repeated as necessary. It also is contemplated that various standard therapies, as well as surgical intervention, may be applied in combination with the application of the agents.

### EXPERIMENTAL

The following examples are provided in order to demonstrate and further illustrate certain preferred embodiments and aspects of the present invention and are not to be construed as limiting the scope thereof.

### EXAMPLE 1

### Materials and Methods

Cell Culture and In Vitro Encapsulation in IKVAV-PA nano-networks. Neural progenitor cells (NPCs) were cultured as previously described (See, e.g., Zhu et al., J. Neurosci. Res. 59, 312 (2000)). Briefly, the cortices of E13 mouse embryos were dissected and plated on un-treated petri dishes in DMEM/F12 media supplemented with bFGF (10 ng/ml). After four days, mechanically and enzymatically dissociated NPCs and undissociated neurospheres (e.g., undissociated NPC aggregates) were plated onto appropriate substrates (e.g., encapsulated in IKVAV-peptide amphiphile (PA), EQS-PA, or alginate gels, or cultured on laminin, poly-D-lysine, or IKVAV peptide coated cover slips). In all cases this was taken as 0 days in vitro.

Encapsulation of NPC in IKVAV- and EQS-PA networks was achieved by first aliquoting 100 μl of PA solution onto a 12 mm cover slip in a 24 well culture plate, forming a self-contained drop. 100 μl of cell suspension in culture media was then pipetted into the drop of PA solution, gentling swirling the pipette tip as the cell suspension was being introduced, forming PA gels. Gels were allowed to sit undisturbed in the incubator (at 37° C. and 5% CO2, with 95% humidity) for >2 hrs., after which 300 μl of NPC culture media was added to the wells, completely submerging the PA gels. Plates were then returned to the incubator. Control 12 mm cover slips were coated with PDL (Sigma, 1 mg/50 ml DMEM) or laminin/PDL (Sigma, 1 mg/100 ml DMEM) and left to sit and dry in a flow hood for >1 hour. Soluble IKVAV peptide was spin coated onto cover slips and allowed to dry overnight. For two dimensional controls, 300 μl of NPC culture media was added to the wells, and 100 μl of NPC cell suspension was aliquoted onto the center of the cover slip, followed by manual shaking of the culture plates to ensure a well distributed cell density. Alginate solutions at 1 wt % were made by mixing 1 g of alginate in 100 ml of physiological buffered saline (PBS) and left on a shaker overnight to allow it to dissolve. 100 μl of 1 wt % alginate was mixed with 100 μl of NPC cell suspension in culture media containing no exogenous calcium (which is normally required to induce alginate gelation), yielding 0.5 wt % alginate gels that would allow direct comparisons with the 0.5 wt % IKVAV-PA experimental gels. The encapsulated NPC in the alginate were returned to the incubator for >2 hours, by which time they had formed weak but stable gels. The culture wells were then filled with 300 μl of culture media, enough to submerge the alginate gels, and returned to the incubator.

Cell Viability/Cytotoxicity Assay. Cell viability/cytotoxicity was assessed by using Molecular Probes LIVE/DEAD cell assay (Molecular Probes). Working concentrations of ethidium homodimer-1 (EthD) and calcein optimized for NPCs were determined as instructed by Molecular Probes, and were determined to be 0.5 and 8 μM, respectively. The culture media was removed from the wells and enough EthD/calcein solution in PBS added to the wells to ensure submersion of the PA gels. Culture plates were returned to the incubator for 20 minutes, and then the EthD/calcein solution removed and the cells washed once with PBS. EthD and calcein fluorescence were imaged using FITC and TRITC filters, respectively, on a Nikon TE-2000 fluorescence microscope.

Immunocytochemistry. The culture media from encapsulated NPCs was removed and the encapsulated cells fixed with 4% paraformaldehyde for 20 minutes at room temperature by submerging the entire PA-gel in fixative. Incubation for 5 minutes with 0.2% Triton-X (toctylphenoxyplyethoxyethanol) was preceded by two washes with PBS. This was followed by another two PBS washes and primary antibody incubation in PBS (anti-β-tubulin III IgG at 1:400 or anti-GFAP at 1:400, Sigma) containing 5% goat or horse serum overnight at 4° C. Following three washes with PBS the cells were incubated with TRITC- or FITC-conjugated secondary antibodies in PBS containing 5% goat or horse serum at room temperature for two hours. Following another three washes with PBS all nuclei were stained with Hoescht's Stain (1:5000, Sigma) for ten minutes at room temperature in order to visualize β-tubulin and GFAP negative cells. Cell imaging was done with a high resolution Cool Snap camera attached to a Nikon TE-2000 fluorescence microscope interfaced with a PC running MetaView imaging software, or an Axiocam camera attached to a Zeiss Axiovert 200 fluorescence microscope interfaced with a PC running AxioVision imaging software.

Cell Counts. Randomly selected fields of view were imaged for different experimental conditions and cells counted using ImageJ (Scion Corporation) morphometric analysis software. Images were checked to make sure there was no bleed-through of fluorescence between filters, and cells semi-automatically counted using ImageJ. Specifically, the total numbers of cells within a given field were counted by manually selecting cells using a marking tool which kept an automatic running count of the total number of cells. Quantitative and statistical analyses of cell counts were done using Matlab (Mathworks) and/or Excel (Microsoft).

Spinal Cord Injection Procedure. Rats were anesthetized using 45 mg/kg Pentobarbital (NEMBUTAL). A laminectomy was performed to expose spinal segment T13 and a stereotaxic micromanipulator (Kopf Instruments) with a Hamilton syringe attached to a 32 gauge needle was used to inject 6 μl at 333 nl/sec of isoosmotic glucose (vehicle) or peptide amphiphile into the spinal cord at T10 at a depth of 1.5 mm. The needle was kept inside the site of injection for 2 minutes after each injection in order to allow the IKVAV-PA to gel without disturbance. Animals injected with peptide amphiphile showed no changes in locomotor behavior or general health, indicating that injection of the peptide amphiphile had no toxic effects.

Intra-ocular Injections. All experiments were done in accordance with the regulations of the Association for Research in Vision and Ophthalmology (ARVO) and Animal Care and Use Committee (ACUC) of Northwestern University. Adult Sprague-Dawley rats (200-250 g) were sacrificed by an overdose of Sodium Pentabarbital or CO2 overdose and their eyes immediately surgically enucleated. A 100 μl Hamilton syringe with a 25 gauge needle was pre-loaded with 80-100 μl of IKVAV-PA solution, and the enucleated eyes placed on the platform of a Nikon SMZ-1000 stereo dissecting microscope. The eyes were manually injected with IKVAV-PA solution into the back of the orbit under the stereo microscope at an oblique angle roughly into the sub-retinal or vitreal spaces, and imaged using the stereo microscope interfaced with a Cool Snap high resolution camera using MetaView imaging software.

Calculation of IKVAV Signal Amplification. The adsorption of proteins at a solid-liquid interface is typically in the vicinity of 1 μg/cm2 (See, e.g., Ratner, Biomaterials Science: An introduction to materials in medicine (Academic Press, San Diego, 1996)). Using this value, and given that the molecular weight of laminin is 800 kDa (See, e.g., Tunggal et al., Microsc. Res. Tech. 51, 214 (2000)), it was calculated that on a two-dimensional surface, such as a glass cover slip or a culture plate, the density of IKVAV epitopes on the surface is

$$\frac{{10}^{- 6} g}{1 {cm}^{2}} \times \frac{mol}{800 \text{,} 000 g} \times \frac{6.023 \times {10}^{23} molecules}{mol} = 7.53 \times {10}^{11} molecules \text{/} {cm}^{2}$$

given that the number of IKVAV epitopes on a native laminin-1 molecule is one.

The density of IKVAV epitopes per square centimeter of a nanofiber surface can also be calculated using known fiber dimensions and molecular modeling. Given that the diameter of a single nanofiber is 7 nm, its circumference is 18.8 nm (C=2πd). Estimating from molecular dimensions that the fiber consists radially of 50 PA molecules, and that 1 cm=107 nm,

$$\begin{matrix}{10}^{7} nm \times \frac{50 PAmolecules}{18.8 nm} = 2.7 \times {10}^{7} PAmolecules \text{/} cm \\ = 2.7 \times {10}^{7} IKVAV \text{/} cm\end{matrix}$$

Assuming that the molecules, being otherwise unconstrained, will not preferentially elongate along one dimension or the other, one can square this to find the number of IKVAV epitopes per square centimeter of nanofiber surface as:
(2.7×10⁷IKVAV/cm)²=7.1×10¹⁴IKVAV/cm²
These two numbers are divided to find the ratio of IKVAV epitopes on a nanofiber to that on a two-dimensional surface, yielding the amplification factor of IKVAV epitopes on a nanofiber relative to a two-dimensional surface of closely packed laminin molecules:

$$\frac{7.1 \times {10}^{14} IKVAV ( PA ) \text{/} {cm}^{2}}{7.53 \times {10}^{11} IKVAV ( lam ) \text{/} {cm}^{2}} \approx {10}^{3} .$$

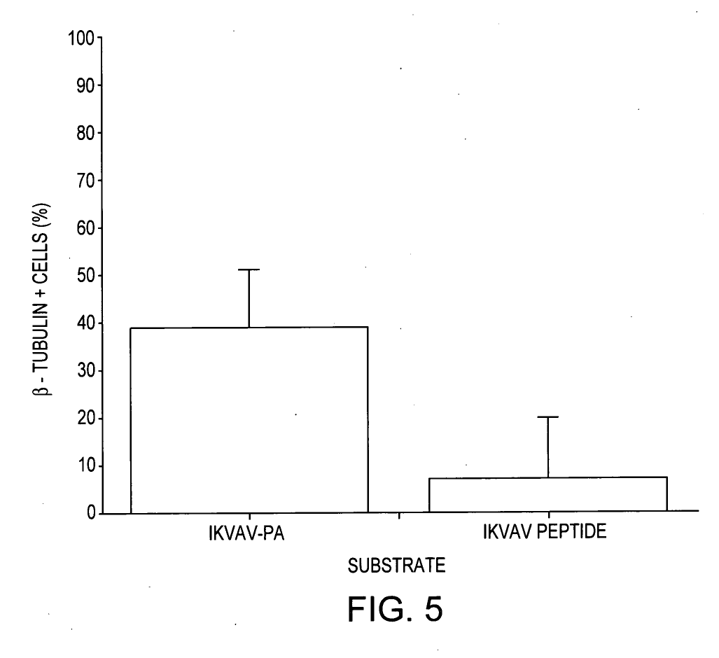

Two-dimensional cultures. For IKVAV peptide experiments, the same 12 mm glass coverslips used for the three-dimensional experiments were soaked in ethanol to encourage hydrophilicity, then spin-coated with 50 μL of a 1 mg/mL IKVAV peptide solution. For IKVAV-PA experiments, the coverslips were coated with PDL (e.g., to encourage adsorption) and subsequently with IKVAV-PA solution. In both cases, the cover slips were allowed to dry overnight and then washed three times with distilled water to remove weakly adherent material before the addition of cell suspension. The results of β-tubulin staining after 1 DIV are shown in FIG. 5.

Mouse spinal cord injuries, amphiphile injections and animal care. All animal care and surgical interventions were undertaken in strict accordance with the Public Health Service Policy on Humane Care and Use of Laboratory Animals, Guide for the Care and Use of Laboratory Animals (Institute of Laboratory Animal Resources, National Research Council, 1996). The Institutional Animal Care and Use Committee approved of all operative procedures. Female, adult 129 SvJ mice (10 weeks old; Jackson Labs, USA) were anesthetized using avertin intraperitoneally. A laminectomy was performed and the spinal cord was compressed dorsoventrally at T10 by the extradural application of a modified Kerr-Lougheed aneurysm clip for 1 min (FEJOTA mouse clip, University Health Network, Canada). The skin was sutured using AUTOCLIP (9 mm, Becton Dickinson). Post-operatively, animals were kept under a heat-lamp to maintain body temperature. A 1.0 cc injection of saline was given subcutaneously which was repeated daily for the first week following the injury. Mice that exhibited any hind-limb movement 24 hours after the injury were excluded from the study. In the event of discomfort, buprenex (2 mg/kg SC, twice daily) was administered. Gentamycin was administered once daily in the event of hematuria (20 mg/kg) subcutaneously once a day for 5 days.

Peptide amphiphile solution or vehicle was injected 24 hours after the spinal cord injury using borosilicate glass capillary micropipettes (Sutter Instruments) (OD: 100 μm). The interior of the pipettes was lined with SIGMACOTE (Sigma) to reduce the surface tension. The capillaries were loaded onto a Hamilton syringe using a female luer adaptor (WPI) which in turn was controlled by a Micro4 microsyringe pump controller (WPI). The amphiphile was diluted 1:1 with a 580 μM solution of glucose just prior to injection and loaded into the capillary. Mice were anesthetized using avertin anesthesia as described above. The autoclips were removed and the incision was reopened exposing the injury site. The micropipette was manually inserted to a depth of 750 μm measured from the dorsal surface of the cord and 2.5 μl of the diluted amphiphile solution or vehicle was injected at the rate of 2.5 μl/min. The micropipette was gradually withdrawn at intervals of 250 μm to leave a trail (ventral to dorsal) of the nanfiber gel in the cord. At the end of injection, the capillary was left in the cord for an additional 5 min, after which the pipette was withdrawn and the wound closed. Post operative care was provided. For all experiments, the experimenters were kept blinded to the identity of the animals.

GFAP quantitation. Following immunostaining, the fluorescence intensity of GFAP immunoreactivity was measured to estimate the fold-increase in GFAP levels around the lesion over baseline levels in uninjured parts of the cord. For each animal, sections at equivalent medio-lateral depth were used for analysis. The sections were then imaged on the Zeiss UVLSM-Meta confocal microscope (Carl Zeiss, Inc., Thornwood, N.Y.). Each confocal scan was performed using identical laser powers, gain and offset values. These values were set such that the pixels in the images of the lesioned area did not saturate. Z stacks of the scans were reconstructed using LSM image browser (Carl Zeiss). Fluorescence quantitation was performed by converting the entire Z-stack into a monochrome (.tif) image and subsequently measuring the gray level of each pixel. Each pixel has a gray scale that ranges from 0 to 255. The total pixel intensity of each stack was integrated using MetaMorph 2.6 software. Intensity values at the lesioned area for each individual section were normalized to the baseline values derived from scans taken over uninjured parts of the section, which were defined as >500 μm away (both rostral and caudal) from the edge of the area of increased GFAP immunoreactivity. For each section, four sites (two rostral and two caudal to the lesion epicenter) in the lesioned area and three in the uninjured area (spanning both grey and white matter) were scanned and the total intensity values averaged for each group. At least four sections were analyzed for each animal in such a manner. The final fluorescence values were expressed as fold increases over the baseline (uninjured area) values for individual sections which were then grouped for each animal for comparison between gel and vehicle-injected groups.

Tract tracing. At 1 day or 9 weeks post injury, mice were anesthetized with Avertin and injected with mini-ruby-conjugated BDA (Molecular Probes, Eugene Oreg.) using a 10 μl Hamilton microsyringe fitted with a pulled glass micropipette. For dorsal column labeling, 2 μl were injected into the L5 dorsal root ganglion. The corticospinal tract was labeled through 3 injections (0.5 μl each) made at 1.0 mm lateral to the midline at 0.5 mm anterior, 0.5 mm posterior, and 1.0 mm posterior to bregma, and at a depth of 0.5 mm from the cortical surface. Animals were sacrificed using CO₂ inhalation 14 days later and perfused.

BDA processing and tract tracing. Floating serial sections were collected and washed 3 times in 1×PBS and 0.1% Triton X-100, incubated overnight at 4° C. with avidin and biotinylated horseradish peroxidase (Vectastain ABC Kit, Vector, Burlingame, Calif.), washed again 3 times in 1×PBS, and then reacted with DAB in 50 mM Tris buffer, pH 7.6, 0.024% hydrogen peroxide, and 0.5% nickel chloride. Sections were then transferred to PBS and mounted in serial order on microscope slides and tracts were traced using Neurolucida software (MicroBrightField, Inc.)

Rat spinal cord injuries, amphiphile injections and animal care. Adult Long Evans Hooded female rats weighing between 150-200 g were anesthetized using pentobarbital anesthesia. Laminectomies were performed and the spinal cords contused at spinal segment T13 with a MASCIS impactor (10 gm weight/50 mm drop which produces the maximal severity of injury). Body temperature and hydration status was maintained as described above. Animals were housed singly to each cage. For the gel injections, 27 gauge needles were used, and the amphiphile was diluted as described above. 24 hours after the contusion injury, rats were re-anesthetized using pentobarbital anesthesia. Following exposure of the injury site, 5 μl of the diluted amphiphile was injected at the rate of 1 μl/min 0.5 mm rostral and caudal to the lesion epicenter at a depth of 1.5 mm. At the end of injection, the needle was left in the cord for an additional 2 min, following which it was withdrawn and the wound closed. Other animals received a similar injection of the vehicle (glucose solution). In the third group (sham injection), the wound was reopened and then closed again without any injection.

Tissue processing and immunohistochemistry. Animals were sacrificed using CO₂ inhalation and transcardially perfused with 4% paraformaldehyde in phosphate buffered saline (PBS). The spinal cords were dissected and fixed overnight in 30% sucrose in 4% PFA. The spinal cords were then frozen in Tissue-Tek embedding compound and sectioned on a Leica CM3050S cryostat. 20 μm thick longitudinal sections were taken. Sections were rinsed with PBS twice and then incubated with anti-GFAP [1:250] (Sigma, mouse monoclonal IgG1) for an hour at room temperature. Following this, sections were rinsed three times with PBS and incubated with alexa-fluor conjugated anti-mouse IgG1 secondary antibodies [1:500] (Molecular Probes) for 1 h at room temperature. Sections were finally rinsed three times with PBS and then incubated with Hoechst nuclear stain for 10 min at room temperature. Following a final rinse with PBS, they were mounted using Prolong Gold anti-fade reagent (Molecular Probes) and imaged using a Zeiss UVLSM-Meta confocal microscope (Carl Zeiss, Inc., Thornwood, N.Y.).

Culture of progenitor cells in the IKVAV-PA and immunocytochemistry. The subventricular zone of P1 (post natal day 1) mice was dissected and grown in DMEM/F12 media supplemented with EGF (20 ng/ml), N2 and B27 supplements, heparin, penicillin, streptomycin and L-glutamine to form floating spheres. Cells were passaged once and the resulting secondary spheres were used for the analysis. Cells were dissociated and plated onto appropriate substrates (e.g. encapsulation in IKVAV-PA or culture on poly-d-lysine/laminin) in DMEM/F12 medium supplemented with EGF (5 ng/ml). In all cases this was taken as 0 days in vitro. Encapsulation of the progenitor cells in IKVAV-PA networks was achieved by first aliquoting 100 μl of PA solution onto a 12 mm cover slip in a 24 well culture plate, forming a self-contained drop. 100 μl of cell suspension in culture medium was then pipetted into the drop of PA solution, with gentling swirling the pipette tip as the cell suspension was being introduced, forming PA gel. The gel was allowed to sit undisturbed in the incubator (at 37° C. and 5% CO2, with 95% humidity) for >2 hrs., after which 300 μl of culture medium was added to the wells, partly submerging the PA gels. Plates were then returned to the incubator. For the control cultures, 12 mm cover slips were coated with Poly D-Lysine for 1 hour, followed by a wash with distilled water and then coated with Laminin (Sigma, 1 mg/100 ml DMEM) overnight. 500 μl of culture medium with cells was added to the wells at a plating density of 5×10⁴ cells/ml. For immunocytochemistry, the culture media from encapsulated cells was removed and the encapsulated cells in the IKVAV-PA were fixed with 4% paraformaldehyde for 20 minutes at room temperature by submerging the entire PA-gel in fixative. For cells plated on laminin, the coverslips were placed in fixative. Incubation for 5 minutes with 0.2% Triton-X was preceded by two washes with PBS. This was followed by another two PBS washes and primary antibody incubation in PBS (anti-β-tubulin III IgG2a at 1:400 or anti-GFAP IgG1 at 1:400, Sigma) containing 5% goat serum overnight at 4° C. Following three washes with PBS the cells were incubated with TRITC- or FITC-conjugated secondary antibodies in PBS at room temperature for 1 hour. Following another three washes with PBS all nuclei were stained with Hoescht's stain (1:5000, Sigma) for ten minutes at room temperature in order to visualize the nuclei all cells including β-tubulin and GFAP negative cells. Cell imaging was performed with an Axiocam camera attached to a Zeiss Axiovert 200 fluorescence microscope interfaced with a PC running AxioVision imaging software (Zeiss).

Rheological measurements of the peptide amphiphiles. Measurements were taken using a Paar Physica Modular Compact Rheometer with a 25 mm parallel plate configuration. Frequency sweeps between 0.1 and 100 Hz were taken for each PA at 3% strain.

### EXAMPLE 2

### Generation of Self-Assembling Scaffolds

Murine neural progenitor cells (NPCs) were used to study in vitro the use of a self-assembling artificial scaffold to direct cell differentiation. NPCs find use in the replacement of lost central nervous system cells (e.g., after degenerative or traumatic insults) (See, e.g., Okano, J. Neurosci. Res. 69, 698 (2002); Storch and Schwarz, Curr. Opin. Invest. Drugs 3, 774 (2002); Mehler and Kessler, Arch. Neurol. 56, 780 (1999); Pincus et al., Neurosurgery 42, 858 (1998)). The molecular design of the scaffold incorporated the pentapeptide epitope isolucine-lysine-valine-alanine-valine (IKVAV), which is found in laminin and is known to promote neurite sprouting and to direct neurite growth (See, e.g., Kam et al., Biomaterials 22, 1049 (2001); Matsuzawa et al., Int. J. Dev. Neurosci. 14, 283 (1996); Powell et al., J. Neurosci. Res. 61, 302 (2000); Cornish et al., Mol. Cell. Neurosci. 20, 140 (2002); Chang et al., Biosens. Bioelectron. 16, 527 (2001); Wheeler et al., J. Biomech. Eng. 121, 73 (1999); Lauer et al., Biomaterials 23, 3123 (2002); Thiebaud et al., Biosens. Bioelectron. 17, 87 (2002).Yeung et al., Neurosci. Lett. 301, 147 (2001)). As a control for bioactivity, a similar molecule lacking the natural epitope was synthesized, replacing it with the non-physiological sequence glutamic acid-glutamine-serine (EQS). These molecules form physically similar scaffolds by self-assembly, but cells encapsulated within the EQS gels did not sprout neurites or differentiate morphologically or histologically.

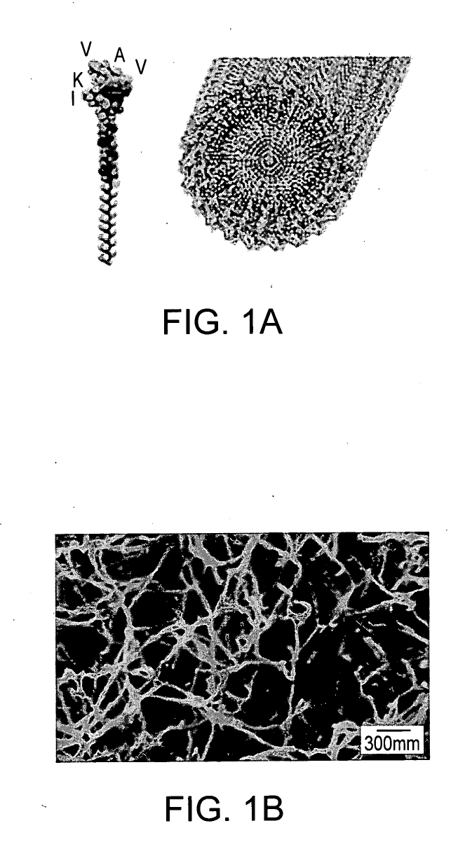

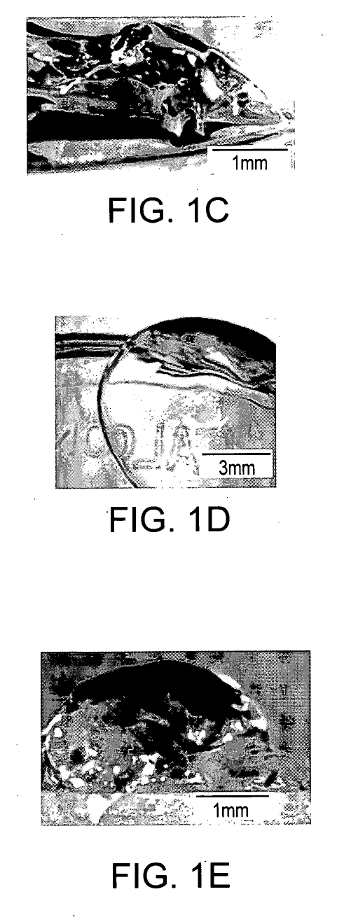

The chemical structure of the IKVAV containing peptide amphiphile (IKVAV-PA) and a molecular graphics illustration of its self-assembly are shown in FIG. 1A, and a scanning electron micrograph of the scaffold it forms is shown in FIG. 1B. In addition to the neurite-sprouting epitope, the molecules contain a Glu residue that gives them a net negative charge at pH 7.4 so that cations in the cell culture medium can screen electrostatic repulsion among them and promote self-assembly when cell suspensions are added. The rest of the sequence consists of four Ala and three Gly residues (A₄G₃), followed by an alkyl tail of 16 carbons. The A₄G₃ and alkyl segments create an increasingly hydrophobic sequence away from the epitope. Although an understanding of the mechanism is not necessary to practice the present invention and the present invention is not limited to any particular mechanism of action, it is contemplated that, in some embodiments, once electrostatic repulsions are screened by electrolytes, the molecules are driven to assemble by hydrogen bond formation and by the unfavorable contact among hydrophobic segments and water molecules.

The nanofibers that self-assemble in aqueous media place the bioactive epitopes on their surfaces at van der Waals packing distances (See, e.g., Hartgerink et al., Science 294, 1684(2001); Hartgerink et al., Proc. Natl. Acad. Sci. U.S.A. 99, 5133 (2002)). These nanofibers bundle to form 3D networks and produce a gel-like solid (See FIGS. 1C, 1D and 1E). The nanofibers have high aspect ratio and high surface areas, 5 to 8 nm in diameter with lengths of hundreds of nanometers to a few micrometers. Nanofibers that form around cells in 3D are able to present epitopes at an artificially high density relative to a natural extracellular matrix. Thus, in some preferred embodiments, the present invention provides a vehicle (e.g., self-assembling scaffold (e.g., comprising nanofibers) for signal (e.g., peptide signal sequence) presentation to cells.

### EXAMPLE 3

### Characterization of Nanofiber Scaffolds

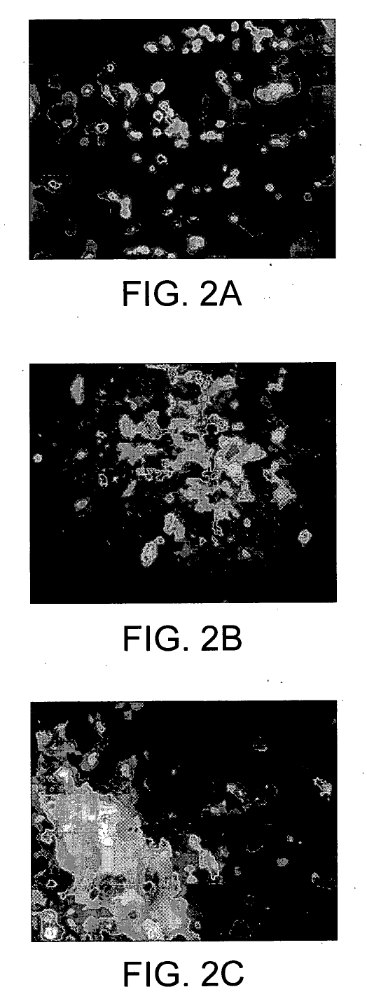

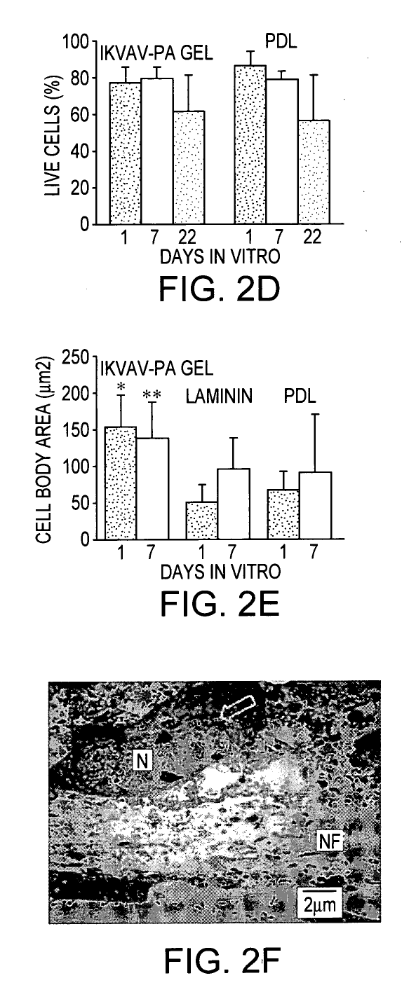

When 1 weight % (wt %) peptide amphiphile aqueous solution was mixed in a 1: 1 volume ratio with suspensions of NPCs in media or physiological fluids, the transparent gel-like solid shown in FIGS. 1C and 1D was obtained within seconds. This solid contained encapsulated dissociated NPCs or clusters of the cells known as neurospheres. The cells survived the self-assembly process and remained viable during the time of observation (22 days) (See FIGS. 2A through 2D). There was no significant difference in viability between cells cultured on poly(D-lysine) (PDL, a standard substrate used to culture many cell types) relative to cells encapsulated in the nanofiber network (See FIG. 2D). Thus, the present invention demonstrates that diffusion of nutrients, bioactive factors, and oxygen through these highly hydrated networks is sufficient for survival of large numbers of cells for extended periods of time. The artificial scaffolds formed by the self-assembling molecules contain 99.5 wt % water, and, although an understanding of the mechanism is not necessary to practice the present invention and the present invention is not limited to any particular mechanism of action, it is contemplated that a high aspect ratio of nanofibers allows a mechanically supportive matrix to form at such low concentrations of the peptide amphiphiles. Thus, the artificial extracellular matrix not only provides mechanical support for cells but also serves as a medium through which diffusion of soluble factors and migration of cells can occur.

### EXAMPLE 4

### Enhanced Differentiation of Neural Precursor Cells Exposed to (e.g., Encapsulated by) Scaffolds

In the bioactive scaffolds, cell body areas and neurite lengths of NPCs that had differentiated into neurons as determined by immunocytochemistry showed statistically significant differences compared to cells cultured on PDL- or laminin-coated substrates. Neurons within the nanofiber networks were noticeably larger than neurons in control cultures. The average cell body area of encapsulated progenitor cells in the networks was significantly greater after 1 and 7 days (See FIG. 2E). Encapsulation in the nanofiber scaffold led to the formation of large neurites after only 1 day (about 57±26 μm, mean ±SD), whereas cells cultured on PDL and laminin had not developed neurites at this early time. The neurons also had significantly longer processes in the scaffolds compared with cells cultured on the PDL substrates after 7 days (P <0.01). There was no statistical difference in neurite length between cells cultured on the PA scaffolds and cells cultured on laminin-covered substrates after 7 days. Transmission electron microscopy (TEM) of NPCs encapsulated in the bioactive scaffold for 7 days showed a healthy and normal ultrastructural morphology, including abundant processes visible in cross section throughout (See FIG. 2F).

### EXAMPLE 5

### Cell Migration Within the Nanofiber Scaffold

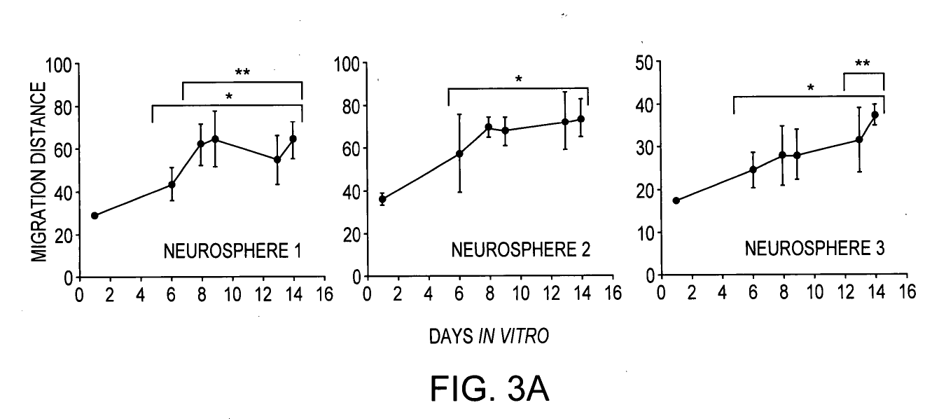

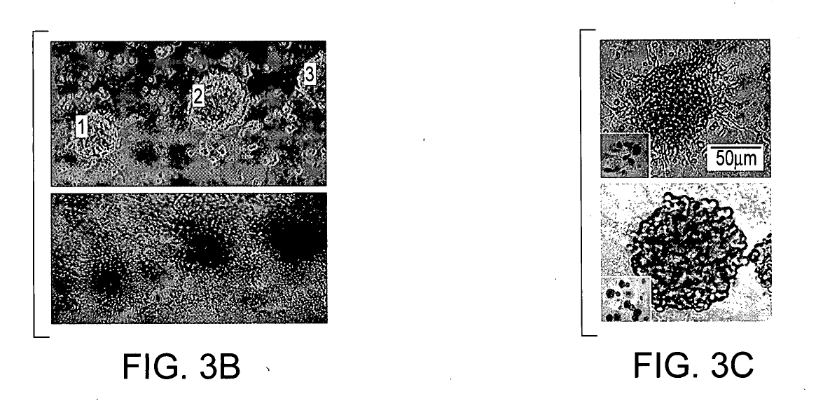

To assess the possibility of cell migration within the nanofiber scaffold, three encapsulated neurospheres were tracked for 14 days (See FIGS. 3A and 3B). All three neurospheres spread out from their centers as constituent cells migrated outward (See FIG. 3B). This effect was quantitated by taking multiple measurements of the distance between the center of each neurosphere and the cell bodies at their outer perimeters (See, e.g., Zhu et al., J. Neurosci. Res. 59, 312 (2000)), and individual cells could be seen to migrate away from the center of the cell mass. Migration of cells within the nanofiber matrix was statistically significant as a function of time (P<0.05) (See FIG. 3A). By contrast, NPCs encapsulated in denser, more rigid networks (98% as opposed to 99.5% water) did not survive. In the nonbioactive scaffolds containing nanofibers with the EQS sequence instead of the bioactive IKVAV sequence, cells failed to migrate away from the neurosphere even though they remained viable. A greater degree of neurite outgrowth was also observed in IKVAV-PA compared to the nonbioactive EQS-PA (See FIG. 3C).

### EXAMPLE 6

### Differentiation of Neural Precursor Cells

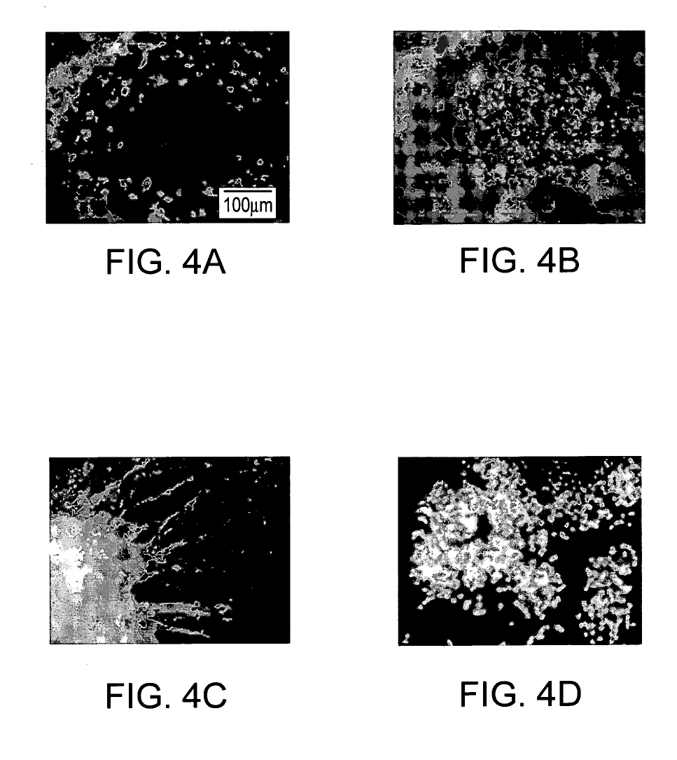

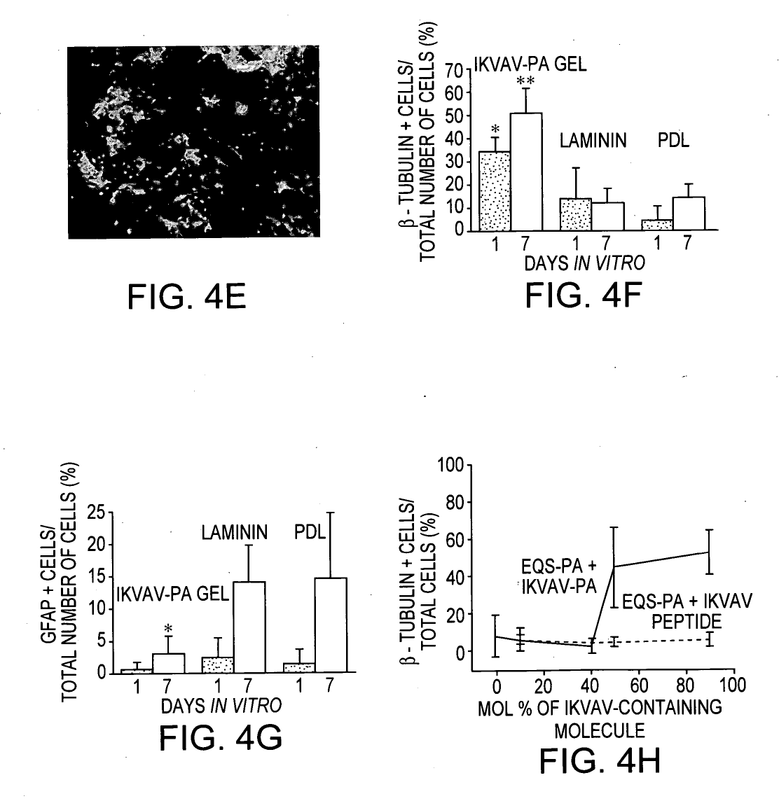

Immunocytochemistry was used to establish the in vitro differentiation of progenitor cells after 1 and 7 days in culture. β-tubulin III and glial fibrillary acidic protein (GFAP) markers for neurons and astrocytes (a subclass of central nervous system (CNS) glia), respectively (See FIGS. 4A through 4E). As shown by immunocytochemistry, NPCs encapsulated in the network with nanofibers presenting IKVAV on their surface differentiated rapidly into neurons, with about 35% of total cells staining positive for β-tubulin after only 1 day. In contrast, there was very little GFAP+ astrocyte differentiation, even after 7 days (<5%). Inhibition of astrocyte proliferation is believed to be important in the prevention of the glial scar, a known barrier to axon elongation following CNS trauma (See, e.g., Rabchevsky and Smith, Arch. Neurol. 58, 721 (2001); Chen et al., Mol. Cell. Neurosci. 20, 125 (2002); Costa et al., Glia 37, 105 (2002)).

Enhanced neuron numbers in the scaffold were detectable after only 1 day in culture and persisted after 7 days. In contrast, GFAP expression was significantly greater in cells cultured on PDL- and laminin-coated substrates relative to cells cultured on nanofiber networks (See FIGS. 4F and 4G). Relative to PDL- or laminin-coated substrates studied previously (See, e.g., Gage et al., Annu. Rev. Neurosci. 18, 159 (1995); Parmar et al., Mol. Cell. Neurosci. 21, 645 (2002); Wu et al., J. Neurosci. Res. 72, 343 (2003); Alsberg et al., Proc. Natl. Acad. Sci. U.S.A. 99, 12025 (2002)), the IKVAV nanofiber scaffold promoted greater and faster differentiation of the progenitor cells into neurons. It was established that the observed differentiation is specific to the IKVAV nanofiber networks by culturing the same cells within scaffolds formed by PA molecules containing the nonbioactive EQS sequence. In these scaffolds and in alginate (a gelatinous compound derived mostly from brown algae that has been well studied as a 3D matrix for various kinds of cells (See, e.g., Canaple et al., J. Biomater. Sci. Polym. Ed. 13, 783 (2002); Chang et al., J. Biomed. Mater. Res. 55, 503 (2001); Marler et al., Plast. Reconstr. Surg. 105, 2049 (2000); Rowley and Mooney, Biomed. Mater. Res. 60, 217 (2002)), the encapsulated cells did not express quantifiable amounts of β-tubulin III or GFAP. As a further test of the 3D EQS control, IKVAV soluble peptide was administered into the EQS-PA-cell suspension mixture at concentrations of 100 μg/ml. Selective neuron differentiation or cells sprouting neurites was not observed. Thus, the present invention demonstrates that the physical entrapment of the bioactive epitope in the self-assembled nanofibers, and not just its presence in the scaffold, is important in the observed cell differentiation.

To determine if the high density of bioactive epitope presented to cells is important in the observed rapid and selective differentiation, “titration” experiments were carried out using networks with varying amounts of IKVAV-PA and EQS-PA. Four different increasing concentrations of the IKVAV-PA were mixed with EQS-PA to form the nanofiber scaffolds containing suspended NPCs as described before. The molar ratios used were 100:0, 90:10, 50:50, 40:60, and 10:90. The presence of nanofibers in these mixed PA networks was verified by TEM. The nanofibers of these networks contained either IKVAV-PA, or EQS-PA, or a mixture of both PA molecules. In either case, a key variable is the density of bioactive epitope in the cell environment.

Immunocytochemistry data in these systems after 1 day (See FIG. 4H) show that the available epitope density around the cells plays a role in the observed neuron differentiation. Cell differentiation in nonbioactive EQS-PA scaffolds was also investigated. In these scaffolds, titration with increasing amounts of soluble IKVAV peptide failed to induce the extent of neuron differentiation observed in IKVAV-PA nanofiber scaffolds (See FIG. 4H), again showing that the presentation to cells of epitopes on the nanofibers is critical to the observed differentiation.

### EXAMPLE 7

### NPC Differentiation on a Two Dimensional (2D) Substrate

NPC differentiation on a two dimensional (2D) substrate coated with IKVAV-PA nanofibers was investigated. The PA molecules self-assemble on surfaces upon drying (See, e.g., Hartgerink et al., Science 294, 1684(2001); Hartgerink et al., Proc. Natl. Acad. Sci. U.S.A. 99, 5133 (2002)), which was verified by TEM. Cells were plated for 1 day on these surfaces, and as shown by immunocytochemistry, the 2D surface was equally effective at inducing differentiation into neurons. Within experimental error, the percentage of cells that differentiated into neurons on the 2D substrates relative to the 3D scaffolds was the same (See FIG. 5). Substrates coated with IKVAV soluble peptide or with laminin (See FIG. 4) did not lead to the significant neuron differentiation observed on IKVAV-PA nanofibers in the same period. The progenitor cells cultured on substrates coated with the IKVAV peptide expressed nearly nonquantifiable amounts of β-tubulin III and/or GFAP during the time of observation. Thus, the present invention demonstrates that nanofibers present to cells a high density of available epitopes that promotes their differentiation either in 2D or 3D cultures. Futhermore, the present invention provides that density, rather than dimensionality of epitope presentation, plays an important role in the rapid and selective differentiation of cells into neurons. An average-sized nanofiber in the network contains an estimated 7.1×10¹⁴ IKVAV epitopes/cm². Closely packed laminin protein molecules in a two-dimensional lattice on a solid substrate have an estimated 7.5×10¹¹ IKVAV epitopes/cm². Thus, nanofibers comprising IKVAV of the present invention amplify the epitope density relative to a laminin monolayer by roughly a factor of 10³.

### EXAMPLE 8

### Self-Assembly of Scaffold in Tissue

The self-assembly of the scaffold can also be triggered by injection of peptide amphiphile solutions into tissue. Ten to 80 μl of 1 wt % peptide amphiphile solutions were injected into freshly enucleated rat eye preparations and in vivo into rat spinal cords following a laminectomy to expose the cord. Thus, these peptide amphiphile solutions can be transformed into a solid scaffold upon contact with-tissues. This process localizes the network in tissue and prevents passive diffusion of the molecules away from the epicenter of an injection site. Furthermore, it is known that animals survive for prolonged periods after injections of the peptide amphiphile solutions into the spinal cord.

Methods of the present invention were also found to be suitable for use in a two dimensional culture. FIG. 5 shows the percentage of total cells that differentiated into neurons in a two dimensional culture on substrates coated with IKVAV-PA nanofibers and substrates coated with IKVAV peptide.

### EXAMPLE 9

### In vivo Model for Characterization of Compositions of the Present Invention

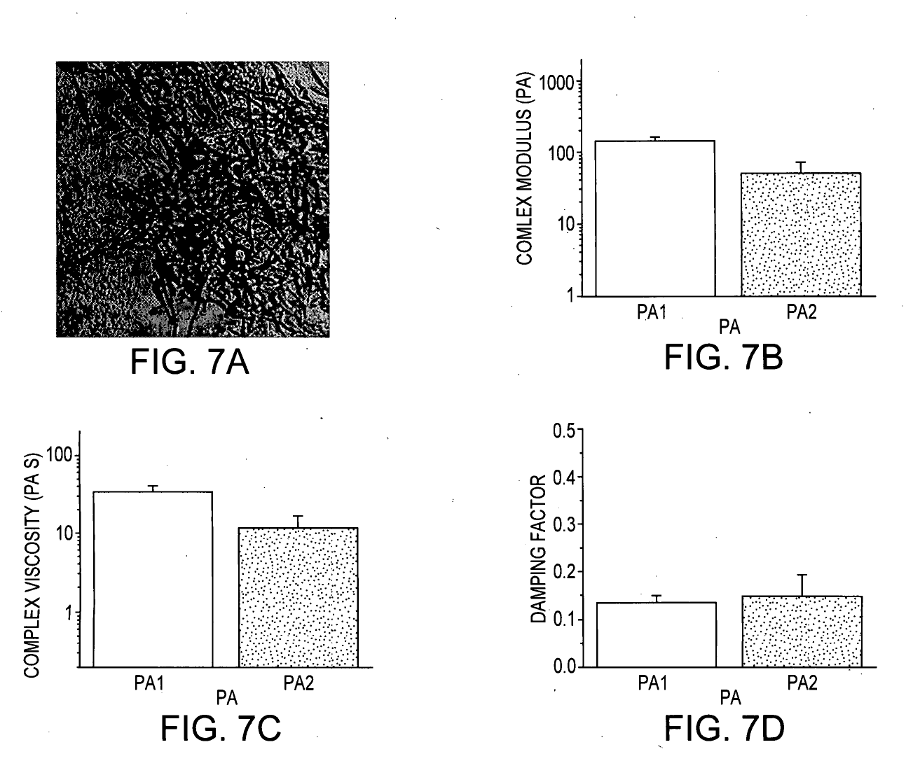

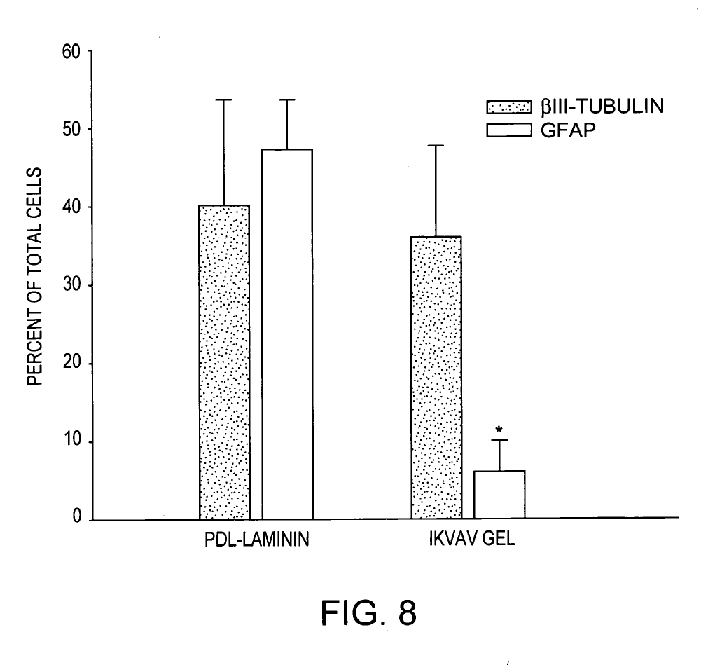

A schematic of some embodiments of peptide amphiphiles generated and characterized during the development of the present invention are presented in FIGS. 6 and 7. As discussed in Examples 1 through 4 above, these biomaterials self-assemble from aqueous solution into three dimensional matrices made up of well defined supramolecular nanofibers designed to be bioactive. Individual nanofibers have a cylindrical shape with a well defined diameter in the range of 6 to 8 nanometers, and are capable of presenting bioactive peptide sequences normal to the fiber axis (See FIGS. 9a-9b, and FIG. 7a). One example of a biomaterial described above forms nanofibers that incorporate the neuroactive pentapeptide epitope isolucine-lysine-valine-alanine-valine (IKVAV) of laminin. These nanofiber matrices were found to promote outgrowth of processes from cultured neurons and to suppress astrocytic differentiation of cultured embryonic and postnatal (See Examples 1-4, and FIG. 8) neural progenitor cells. Thus, experiments were conducted during the development of the present invention in order to determine whether injection of a composition of the present invention (e.g., comprising a peptide amphiphile (e.g., formulated in an aqueous solution)) after nerve injury (e.g., spinal cord injury) to a site of damaged nerve tissue could promote neuronal axon outgrowth and/or reduce astrogliosis and formation of the glial scar, a major impediment to axonal regeneration after spinal cord injury.

The clip compression model of spinal cord injury (SCI) has been used to provide a consistent injury in rodents. This model produces an injury where an initial impact is followed by persistent compression analogous to what is seen in most cases of human SCI (See, e.g., Joshi and Fehlings, J Neurotrauma 19, 191-203 (2002); Joshi and Fehlings, J Neurotrauma 19, 175-190 (2002)). Studies conducted during the development of the present invention used a severe injury (24 g weight) in mice that results in little or no functional weight-bearing hind-limb movement, even in chronic stages following the injury. Adult (10 weeks old) 129SvJ female mice were anesthetized and the clip was applied as described (See, e.g., Joshi and Fehlings, J Neurotrauma 19, 175-190 (2002)) at the T13 level of the thoracic cord resulting in complete paralysis of both hind-limbs. Mice that exhibited any hind-limb movement 24 hours after the injury were excluded from the study.

### EXAMPLE 10

### In vivo Characterization of Peptide Amphiphile

In order to evaluate the stability of the biodegradable peptide amphiphile in the injured spinal cord, a fluorescent derivative was synthesized (See FIG. 6a) to enable visualization of the scaffold within the cord by excitation with light in the UV range. The amphiphile was also modified in order to create a system that gelled more slowly during injections in vivo (See FIGS. 6 and 7). A 1% peptide amphiphile solution in isotonic glucose was injected into the lesion site 24 hours following the injury using a glass capillary micropipette. The fluorescent gel formed within the cord as shown in FIG. 9c (imaging performed 24 hours after injection), and remnants could still be seen in the injured cord 5 weeks after injection (See FIG. 9d). At 5 weeks after injection the fluorescent gel has begun to diffuse within the cord away from the site of injection (e.g., although an understanding of the mechanism is not necessary to practice the present invention and the present invention is not limited to any particular mechanism of action, this may reflect biodegradation of the peptide and lipid-based matrix). Thus, the present invention demonstrates that the lifetime of the nanofiber scaffold in vivo is on the order of weeks.

### EXAMPLE 11

### Nanofiber Scaffold Effect on Astrogliosis

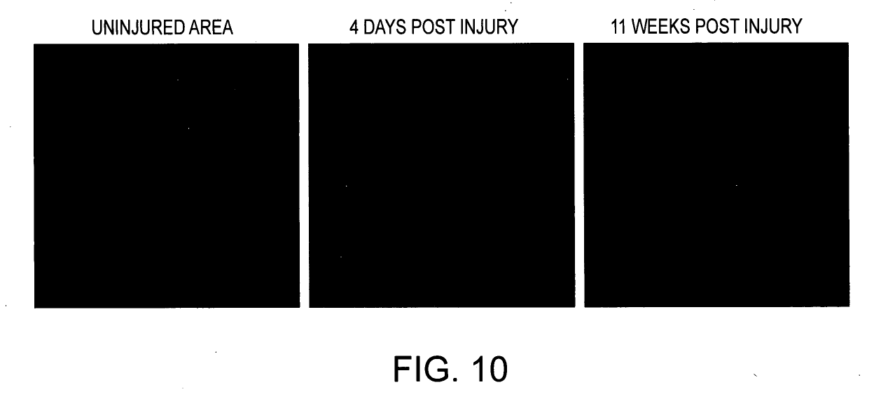

Astrogliosis following neural injury involves an early hypertrophic (increased cell size) as well as a later hyperplastic (increased cell number) response (See, e.g., Faulkner, J. R. et al., J Neurosci 24, 2143-55 (2004); Steward et al., J Comp Neurol 459, 1-8 (2003); and FIG. 10). To analyze the effect of the gel on astrogliosis, 24 hours after spinal cord injury the lesion site was injected either with the peptide amphiphile diluted 1:1 with glucose or with vehicle (glucose, See Example 1). The treatment was delayed until 24 hours after the injury to make the findings clinically relevant (e.g., in order to represent events that may occur to a human subject). Vehicle-injected animals were used as the control group as experiments demonstrated that the vehicle-injected group had slightly enhanced recovery compared to sham operated animals (See FIG. 10b). For this and all subsequent experiments, the experimenters were kept blinded to the identity of the animals.

Mice were sacrificed either at 4 days or 11 weeks after the injury for immunohistochemical analysis. To quantify the astrogliosis, the intensity of GFAP immunofluorescence around the lesion was measured compared to baseline levels in uninjured parts of the cord (See Example 1 for methodology). At 4 days after SCI, GFAP immunohistochemistry revealed no obvious differences between the treated and control animals, and quantitation of the levels of GFAP at that time similarly revealed no difference (See FIG. 9f). Thus, at this initial stage the nanofiber gel did not prevent astrocytic hypertrophy. However, 11 weeks after the injury GFAP immunohistochemistry revealed an obvious reduction in the number of astrocytes in the gel-injected group (See FIG. 9e), and quantitation of GFAP immunofluorescence (FIG. 9f) showed a significant reduction in the treated group (means: 2.0±0.1 for gel-injected group; 2.7±0.2 for vehicle injected group, p<0.04). Thus, the present invention demonstrates that injection of the peptide amphiphile solution suppressed astroglial proliferation and scar formation at the lesion site, while leaving unaltered the initial reactive hypertrophy important for repairing the blood brain barrier and for restoring homeostasis (See, e.g., Faulkner, J. R. et al. J Neurosci 24, 2143-55 (2004).

### EXAMPLE 12

### In vivo Administration of Nanofiber Gel Promotes Rejuvenation of Injured Motor and Sensory Axons

It was next determined whether administration of compositions of the present invention could promote actual regeneration of injured motor and sensory axons. For analysis of descending corticospinal motor fibers (CST), biotinylated dextran amine (BDA) was injected into sensorimotor cortex at 9 weeks after the injury (See Example 1 for methodology). Two weeks later, the animals were sacrificed and the tissue was processed from 3 animals in each group for analysis of serial 20 μm thick longitudinal sections. Serial sections were collected so that individual axons could be traced from section to section, and the course was traced using Neurolucida imaging software for each axon that was labeled within a 500 μm distance rostral to the lesion. Representative traces from a gel-injected and a vehicle-injected animal illustrate the marked difference between the treated and control groups that received BDA injections 9 weeks after the injury (analyzed at 11 weeks) (See FIG. 11). Almost 80% of labeled axons in the nanofiber gel group entered the lesion compared to about 50% of the fibers in control animals (See FIG. 11). No fibers in the control animals were ever detected as far as 25% of the way across the lesion. By contrast, approximately 50% of the fibers in the nanofiber gel treated group penetrated half of the way through the lesion, and about 45% of the fibers penetrated three quarters of the distance. Strikingly, about 35% of the fibers actually grew through the lesion and entered the spinal cord caudal to the lesion. With the severity of injury that was used, sparing of axons would be unlikely.

Nevertheless, to more definitely exclude the possibility of axon sparing, BDA was injected into sensorimotor cortex at 1 day after the injury and tract tracing was examined 2 weeks later. At this time only 15% of the fibers in the gel-treated and no fibers in the vehicle-treated group were observed entering the lesion. More importantly, at this time no fibers in either group were ever observed even 25% of the way through the lesion, demonstrating that spared fibers were not present. Additionally, it is also important to apply rigorous criteria for distinguishing regeneration from sparing of axons (See, e.g., Steward et al., J Comp Neurol 459, 1-8 (2003). Axons in the nanofiber gel-treated group entered the tissue of the scar and followed an unusual course through the tissue environment. The distance traveled by the axons was consistent with plausible regeneration rates, and, as shown in the traces, the fibers stopped shortly past the lesion. Unusual branching patterns and other atypical morphological characteristics in these axons were also observed. Thus, the present invention demonstrates that nanofiber gel promotes regeneration of corticospinal tract motor fibers.

For analysis of sensory axon regeneration, BDA was injected into the L5 dorsal root (sensory) ganglion, using the entry point of the sciatic nerve as a landmark, nine weeks after the injury, and the animals sacrificed 2 weeks later for analysis. Serial sectioning and tracing of axons using Neurolucida imaging was performed for 4 animals in each group in a manner analogous to the tracing of descending motor fibers. Traces from a gel-injected and a vehicle-injected animal illustrate the marked difference between the treated and control groups that received BDA injections 9 weeks after the injury (See FIG. 12). Approximately 60% of labeled axons in the gel group entered the lesion compared to only about 20% of the fibers in control animals (See FIG. 12). Only rare fibers in the control animals grew 25% of the way across the lesion, and no fibers in control animals penetrated as far as 50%. By contrast, approximately 35% of the fibers in the treated group penetrated 25% of the way through the lesion, and about 25% of the fibers penetrated 50% of the distance. Importantly, about 10% of the fibers actually grew through the lesion and entered the spinal cord rostral to the lesion. These axons also met the criteria for regenerated versus spared axons (See, e.g., Steward et al., J Comp Neurol 459, 1-8 (2003).

In order to more definitely exclude the possibility of axon sparing, BDA was injected into the L5 dorsal root (sensory) ganglion 1 day after the injury and tract tracing was examined 2 weeks later. At this time, no fibers in either group were ever observed penetrating even as far as 25% of the way through the lesion, demonstrating that spared fibers were not present. Thus, the present invention demonstrates that the bioactive nanofiber network promoted regeneration of sensory as well as corticospinal tract motor fibers.

### EXAMPLE 13

### Anatomical Improvements are Associated with Behavioral Recovery

It was determined whether the observed anatomical improvements were associated with behavioral recovery using the Basso, Beattie, and Bresnahan (BBB) locomotor scale modified for the mouse (See, e.g., Joshi and Fehlings, J Neurotrauma 19, 175-190 (2002); Bresnahan et al., Exp Neurol 95, 548-70 (1987); Basso et al., Exp Neurol 139, 244-56 (1996)). Behavioral testing was performed weekly for 9 weeks following treatment (FIG. 13a). For the first 5 weeks there were no distinguishable differences between the control and the group injected with the self-assembling molecules, but at 5 weeks and thereafter the gel-injected group displayed significant behavioral improvement compared to the control group. At 9 weeks the mean BBB score for the control group was 7.03+0.8 while the mean score for the gel-injected group was 9.2±0.5 (p<0.04). This represents significant functional recovery, as a score of 7 implies no functional movement despite an extensive range of movement in all three joints in the hind limb, whereas a score of 9 indicates dorsal stepping in which the animal steps on the dorsal side of its foot during locomotion (e.g., the hind-limb movement has a functional use). Notably the difference between the groups was apparent in the later stages following the injury, more consistent with a regenerative response than a protective effect.

The effects of the bioactive gel on behavioral recovery was also evaluated using the BBB locomotor scale in a different, more widely used contusion model of spinal cord injury, the MASCIS impactor in rats (See, e.g., Young, Prog Brain Res. 137:231-55 (2002)) (See FIG. 13b). The results were similar to findings with the clip contusion model in mice. The vehicle-injected and gel-injected groups were nearly indistinguishable from each other for the first 5 weeks. There was also no significant difference between these groups and a sham-injected group although the injected groups both trended towards slightly higher scores. At 5 weeks and thereafter the gel-injected group displayed significant behavioral improvement compared to both the sham injected and the vehicle-injected groups. At 9 weeks the mean BBB score for the sham injected control was 9.4±0.6 while the mean score for the vehicle-injected group was 9.9+0.5. By contrast, the mean BBB score for the gel-injected group was 12.7+0.6 which was significantly higher than either control group (p<0.02). Functionally this is a striking improvement and represents the difference between dorsal stepping in the control groups versus consistent weight-supported plantar steps with frequent front-limb hind-limb coordination in the group with the self-assembling bioactive matrix.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method of altering development of a neuron comprising contacting said neuron with a composition comprising a peptide amphiphile.

2. The method of claim 1, wherein said altering development of a neuron comprises axonal growth.

3. The method of claim 2, wherein said axonal growth comprises descending motor fiber growth.

4. The method of claim 2, wherein said axonal growth comprises ascending sensory fiber growth.

5. The method of claim 1, wherein said peptide amphiphile comprises an IKVAV (SEQ ID NO: 1) sequence.

6. The method of claim 1, wherein said composition comprising a peptide amphiphile further comprises a neurotrophic factor.

7. A method for treating a subject comprising:
a) providing a subject with a damaged nerve, and
b) administering a composition comprising a peptide amphiphile to said subject under conditions such that neuron growth occurs in said subject.

8. The method of claim 7, wherein said neuron growth comprises descending motor fiber growth.

9. The method of claim 7, wherein said neuron growth comprises ascending sensory fiber growth.

10. The method of claim 7, wherein said neuron growth is accompanied by reduced astrogliosis in said subject.

11. The method of claim 7, wherein said damaged nerve is a nerve in a spinal cord that has been damaged.

12. The method of claim 7, wherein said damaged nerve comprises a damaged sensory neuron and/or a damaged motor neuron.

13. The method of claim 7, wherein said administering comprises parenteral administration of an aqueous solution comprising said peptide amphiphile.

14. The method of claim 13, wherein said peptide amphiphile forms a nanofiber gel upon contact with said damaged nerve.

15. The method of claim 7, wherein said composition comprising a peptide amphiphile is co-administered with one or more other agents.

16. The method of claim 15, wherein said one or more other agents are selected from the group consisting of a neurotrophic factor, an inhibitor of a neuronal growth inhibitor, a neuronal growth attractant and a neuronal growth inhibitor.

17. The method of claim 16, wherein said neurotrophic factor is selected from the group consisting of Nerve growth factor (NGF), Brain-derived neurotrophic factor (BDNF), Neurotrophin-3 (NT-3), Neurotrophin-4/5 (NT-4/5), Ciliary neurotrophic factor (CNTF), Leukemia inhib. factor=chol. neuronal diff. factor (LIF/CDF), Cardiotrophin-1, Basic fibroblast growth factor (bFGF), Acidic fibroblast growth factor (aFGF), Fibroblast growth factor-5 (FGF-5), Insulin, Insulin-like growth factor I (IGF-I), Insulin-like growth factor Ii (IGF-II), Transforming growth factor β1 (TGFβ1), Transforming growth factor β2 (TGFβ2), Transforming growth factor β3 (TGFβ3), Activin, Glial cell-derived neurotrophic factor (GDNF), MidkineHeparin-binding neurotrophic factor (HBNF), Pleiotrophin, Epidermal growth factor (EGF), Transforming growth factor α (TGFα), Schwannoma-derived growth factor, Heregulin (neuregulin, ARIA), Interleukin 1, Interleukin 2, Interleukin 3, Interleukin 6, Axon ligand-1 (Al-1), elf-1, ehk1-L, and LERK2.

18. A pharmaceutical composition comprising a peptide amphiphile comprising an IKVAV (SEQ ID NO: 1) sequence, wherein said composition is configured to alter neuron growth in a subject.

19. The composition of claim 18, wherein said peptide amphiphile comprises a SLSL (SEQ ID NO: 2) sequence and/or a A3 sequence.

20. The composition of claim 18, wherein said peptide amphiphile comprises a heteroatom.


SEQUENCE LISTING (14 sequences)

SEQ ID NO: 1 - protein, 5 aa, Artificial Sequence
  Description of Artificial Sequence Synthetic peptide
IKVAV

SEQ ID NO: 2 - protein, 4 aa, Artificial Sequence
  Description of Artificial Sequence Synthetic peptide
SLSL

SEQ ID NO: 3 - protein, 5 aa, Artificial Sequence
  Description of Artificial Sequence Synthetic peptide
YIGSR

SEQ ID NO: 4 - protein, 5 aa, Artificial Sequence
  Description of Artificial Sequence Synthetic peptide
YGSIR

SEQ ID NO: 5 - protein, 7 aa, Artificial Sequence
  Description of Artificial Sequence Synthetic peptide
AAAAGGG

SEQ ID NO: 6 - protein, 13 aa, Artificial Sequence
  Description of Artificial Sequence Synthetic peptide
AAAAGGGEIKVAV

SEQ ID NO: 7 - protein, 13 aa, Artificial Sequence
  Description of Artificial Sequence Synthetic peptide
AAAAGGGEYIGSR

SEQ ID NO: 8 - protein, 13 aa, Artificial Sequence
  Description of Artificial Sequence Synthetic peptide
AAAAGGGKYIGSR

SEQ ID NO: 9 - protein, 13 aa, Artificial Sequence
  Description of Artificial Sequence Synthetic peptide
AAAAGGGKIKVAV

SEQ ID NO: 10 - protein, 13 aa, Artificial Sequence
  Description of Artificial Sequence Synthetic peptide
SLSLAAAEIKVAV

SEQ ID NO: 11 - protein, 13 aa, Artificial Sequence
  Description of Artificial Sequence Synthetic peptide
SLSLAAAEIKVAV

SEQ ID NO: 12 - protein, 13 aa, Artificial Sequence
  Description of Artificial Sequence Synthetic peptide
AAAAGGGKFIGSR

SEQ ID NO: 13 - protein, 13 aa, Artificial Sequence
  Description of Artificial Sequence Synthetic peptide
AAAAGGGKYIGSR

SEQ ID NO: 14 - protein, 13 aa, Artificial Sequence
  Description of Artificial Sequence Synthetic peptide
AAAAGGGEIKVAV